(19) United States
(12) Patent Application Publication
(10) Pub. No.: US 2010/0143376 A1
(43) Pub. Date: Jun. 10, 2010

(54) Antibodies Specific for Rubella Virus

(75) Inventors: Roberto Burioni, Rimini (IT); Massimo Clementi, Lesi (IT)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: RIBOVAX BIOTECHNOLOGIES S.A., PETIT-LANCY (CH)

(21) Appl. No.: 12/530,030

(22) PCT Filed: Mar. 6, 2008
(86) PCT No.: PCT/EP08/01791

§ 371 Date: Feb. 11, 2010

(30) Foreign Application Priority Data
Mar. 6, 2007 (EP) 07004573.7

(51) Int. Cl. A61K 39/42; C07K 14/47; C07K 16/00; C07H 21/00; C12N 15/63; C12N 7/00; C12N 1/00; C12N 5/00; C12Q 1/70; C12P 21/00; A61P 31/12

(52) U.S. Cl. 424/159.1; 530/350; 530/387.1; 536/23.5; 435/320.1; 435/235.1; 435/243; 435/325; 435/5; 435/71.1

(57) ABSTRACT

The present invention provides novel antibody sequences that bind and neutralize Rubella Virus (RuV). The novel sequences can be used for the medical management of RuV infection, in particular for detecting the virus or for preparing pharmaceutical compositions. The RuV-specific antigens recognized by such antibody sequences can be identified using novel phage libraries that display peptides.

### FIELD OF THE INVENTION

The invention relates to novel antibody sequences isolated from phage display libraries having binding and neutralizing activities specific for a virus.

### BACKGROUND OF THE INVENTION

Phage display technologies take advantage of the small dimension and the adaptability of the genome of filamentous phage (such as M13) that infect bacterial cells (e.g. Escherichia coli cells) for cloning, selecting, and engineering polypeptides (antibody fragments, bioactive peptides, enzymes, etc.) that are expressed on their surface and can exert biological functions following their interaction with a target.

Several cloning and expression strategies, vectors, libraries, methods for propagating phage, and screening assays have been developed for different applications, as reviewed in articles (Bradbury A and Marks J, 2004; Mancini N et al., 2004; Conrad U and Scheller J, 2005; Hust M and Dubel S, 2005), and books (“Phage display: A Practical Approach”, vol. 266, ed. Clackson and Lowman H, Oxford Univ. Press, 2004; “Phage Display: A Laboratory Manual”, ed. Burton D et al., CSHL Press, 2001).

A phage display library is formed by a population of recombinant phage, each displaying a single element of a repertoire of protein sequences on its surface. Phage that express specific proteins can be isolated from the library by iterative affinity-based and/or activity-based selection processes (the “panning”). For example, the proteins can be antibody fragments, in the form of variable heavy/light chain heterodimers (commonly named as Fabs) or single chain Fragment variable (scFv), that can be isolated and characterized on the basis of their specific binding affinity for antigens or activity in biological and functional assays.

In particular, screening processes have been developed to identify antibody fragments that have high affinity and specificity for pathogens and biological targets, sometimes with relevant biological activity associated to such binding properties. In fact, an entire therapeutic approach (named passive immunotherapy or passive serotherapy) has been built on the antigen-binding features of antibodies and antibody fragments directed against human or non-human therapeutic targets (Dunman P and Nesin M, 2003; Keller M and Stiehm E, 2000). Passive immunotherapy consists of the administration to individuals of pharmaceutical compositions comprising therapeutic antibodies with a defined binding specificity for a pathogenic antigen (a toxin, a human protein, a virus, or a parasite, for example).

Passive immunotherapy has been introduced into clinical practice, rapidly expanding the opportunities for the treatment of a wide variety of diseases (including infectious diseases, immune-mediated diseases and cancer). This approach can be particularly effective in patients whose immune system is unable to produce them in the amounts and/or with the specificity that are required to block and/or eliminate the targeted molecule (Chatenoud L, 2005; Laffly E and Sodoyer R, 2005).

Among pathogenic antigens that can be targeted using therapeutic antibodies, viruses that infect human cells are of particular importance. The administration of such antibodies can inhibit the propagation of the virus in the patient, and potentially block the outbreak of a viral infection in the population. Alternatively, the antibody may be administered to a patient having a weakened immune system for a more or less prolonged period of time (e.g. immunosuppressed, elderly, or transplanted individuals) that become much more sensitive to infectious diseases, including those that normally do have not serious and/or permanent consequences on health of immunocompetent individuals.

Rubella Virus (RuV, German Measles) is an example of such viruses. RuV is member of the Togaviridae family presenting an RNA genome, two non-structural proteins, and a virion envelope formed by three viral structural proteins (E1, E2, and C) that are combined to host-derived lipid bilayer (Banatvala J and Brown D, 2004; Lee J and Bowden D, 2000; “Rubella Viruses” Perspective in Medical Virology, Ed. Banatvala J and Peckham C, vol. 15, 2006, Elsevier).

RuV is present in nasopharyngeal secretions, blood, faeces, and urine of infected individuals and it is transmitted from person to person via respiratory aerosols. Ruv is a neurotropic virus, that primarily infects neural cells (such as oligodendrocytes), but RuV strains vary in their abilities to infect, replicate, and persist in various cell types, in particular neural and joint tissue (such as synovial cells). The viral arthrotropism may explain the association of RuV infection with joint symptoms (Lund K and Chantler J, 2000; Masuko-Hongo K et al., 2003). RuV induces cytopathic effects, apoptosis, and the arrest of cell cycle in infected cells. In fact, RuV proteins modify host cell signaling and metabolism by interfering with protein-protein interactions, gene expression, and protein phosphorylations (Hofmann J et al., 1999; Cooray S et al., 2005; Atreya C et al., 2004; Domegan L and Atkins G, 2002; Adamo M et al., 2008; Figuereido A et al., 2000).

RuV is generally responsible for a mild illness, with low-grade fever and rash appearing 16 to 20 days after exposure and appearing mainly on the face and the extremities. RuV infection may also be entirely asymptomatic. RuV infection only rarely causes complications in adults, such as post-infectious encephalopathy, thrombocytopenic purpura, hamorragic manifestations, or arthritis (Banatvala J and Brown D, 2004). RuV infection is also involved in a series of ocular disorders such as Fuchs heterochromic iridocyclitis (De Groot-Mijnes J et al., 2006).

However, RuV infection becomes a major concern when the infected patient is a pregnant woman, in particular in developing countries and in immigrant populations. RuV has the ability to cross the placental barrier and infect fetal tissues, even though maternal infection is not always followed by fetal infection. RuV infection during the first 12 weeks of gestation leads in 80-90% of cases to fetal death or to a variety of different anomalies, commonly grouped under the definition of Congenital Rubella Syndrome (CRS), such as cardiac and ocular defects, hearing loss, and mental retardation. The risks decrease if the infection appears in the following weeks of gestation but fetal damages due to viral teratogenesis may be still present (Hinman A et al., 2002; Lee J and Bowden D, 2000; Atreya C et al., 2004). Moreover, the serodiagnostic assays that allow distinguishing antibodies due to a recent RuV infection from false positives or those due to antibodies persisting many months after infection or vaccination, are not fully reliable (Andrews J, 2004; Mendelson E et al., 2006).

RuV vaccination is widely established in the industrialized world and is highly effective in reducing CRS and RuV-induced fetal death. Nonetheless, the genetic characteristics of wild-type RuV genomes in recent outbreaks are still attentively surveyed by World Health Organization for early intervention. This surveillance involves the definition of reference RuV genotypes and strains and the related diagnostic assays, together with analysis and classification of infective and genetic properties of RuV strains identified in outbreaks worldwide (WHO 2005; WHO 2006; Reef S et al., 2002; Zheng D et al., 2003).

In fact, several studies showed that a non-negligible percentage of individuals in developed countries (at least 5%) has low or undetectable antibody concentrations against RuV. These subjects have been detected in both populations following several months or years from their vaccination and in recent immigrants or refugees who had limited access to vaccination programs. Thus, there is quite large number of candidates for additional treatment against RuV infection due to the waning of specific immunity, in particular girls approaching puberty and to women of childbearing age in order to prevent RuV infection and the possibility of CRS (Pebody R et al., 2000; Nicoara C et al., 1999; Matter L et al., 1997; Ki M et al., 2002; Ushida M et al., 2003; Banerji A et al., 2005; Rota M et al., 2007; Mendelson E et al., 2006). Epidemiological studies suggest the importance of establishing programs for RuV routine childhood and adults vaccination, surveillance, and selective reimmunization (Greenaway C et al., 2007; Kremer J et al., 2007; Semerikov V et al., 2000; Banatvala J and Brown D, 2004; Chakravarti A and Jain M, 2006; Am. Acad. Pediatr. Com. Infect. Dis., 2007).

RuV pathogenesis and immunobiology has been studied in connection to the mechanisms and the efficacy of the immune response to RuV infection and vaccination in humans and in animal models. Cell-based models for RuV infection have been established (Cusi M et al., 1995; Duncan R et al., 1999; Garbutt M et al., 1999; Cordoba P et al., 2000a). Very sensitive tests for the early detection of a primary RuV infection and of the RuV-specific immune response have been developed (Takahashi S et al., 1998; Tzeng W et al., 2005; Giessauf A et al. 2004; Wilson K et al. 2006). However, vaccination may take several weeks or months to mount an effective immune response and the fetus is be still vulnerable to RuV infection during that time. RuV infection can be dangerous in immunosuppressed patients, in whom the use of vaccines is not always advisable.

Chemical agents that may be useful against RuV are still in the early phase of development (Mugnaini C et al., 2007). However, it is known that chemotherapy and antiviral therapies can significantly lose immune protection to viruses such as RuV and a revaccination ma be required (Yu J et al., 2007; Bekker V et al., 2006; Van Tilburg C et al., 2006; Nilsson A et al., 2002). Preparations of human antibodies having significant anti-RuV titers can be administered to treat or prevent the infection. (Keller M and Stiehm E, 2000; Krause I et al., 2002; Kawamura N et al., 2000), but the efficacy of a similar approach would be improved by using more specific immunological products to neutralize the virus, such as recombinant antibody fragments or monoclonal antibodies.

Human antibody responses directed against RuV, and in particular against the structural proteins of the virus have been assessed using the sera of individuals vaccinated against RuV or infected by wild RuV strains, showing substantial differences in the specificity and in the neutralizing properties of the response in different subjects (Zhang T et al., 1992; Mauracher C et al., 1992; Mitchell L et al., 1992; Zrein M et al., 1993; Mitchell L et al., 1993, Thomas H et al., 1993; Banerji A et al., 2005). Hemagglutinating activity and different antibody neutralization domains were assigned to the structural proteins E1 and E2 (Mendelson E et al., 2006; Cordoba P et al., 2000a; Green K and Dorsett P, 1986). RuV immunopathologies were also studied in animal models using severe combined immune deficient (SCID) mice implanted with tonsil fragments from RuV (Perrenoud G et al., 2004) or mice infected with RuV via the abdominal cavity (Wang Z et al., 2003).

Several murine monoclonal antibodies capable of binding and (in a few cases) of neutralizing RuV, have been generated. The antibodies have been tested with different RuV-related targets (virus-like particles, protein extracts, recombinant proteins, peptides, cells infected in cell culture conditions) for studying viral infection and replication in the cells, as well as the immunogenic epitopes recognized by the antibodies and the neutralizing activities (Green K and Dorsett P, 1986; Terry G et al., 1988; Wolinsky J et al., 1991; Chaye H et al., 1992; Claus C et al., 2006; Qiu Z et al., 1994; Ou D et al., 1992; Wolinsky J et al., 1993; Robinson K et al., 1995; Cordoba P et al., 2000a; Cordoba P et al., 2000b; Cordoba P et al., 1997; Starkey W et al., 1995; Giessauf A et al., 2005; Orellana A et al., 1999; Lee J et al., 1999; Hofmann J et al., 2000; EP299673; WO 93/14206; WO 91/02748; WO 95/09232).

Very few human monoclonal anti-RuV antibodies, none of them with relevant anti-RuV neutralization activities, have been reported in the literature. Such antibodies have been identified using human B cells that have been immortalized by cell fusion and/or EBV infection (WO 07/011,698, see clone R335.6.4; Steenbakkers P et al., 1992; Hilfenhaus J et al., 1986). Human Fab antibody fragments against RuV have been identified (Williamson R et al., 1993).

Although vaccination has brought major improvements, the disease persists. The identification and production of novel human antibodies and antibody fragments that efficiently bind and neutralize RuV is still of particular importance for establishing improved treatments for the diagnosis, therapy and/or prevention of this infectious disease in the population.

### SUMMARY OF THE INVENTION

The present invention provides novel antibody sequences that bind and neutralize RuV, and that can be used for detecting, treating, inhibiting, preventing, and/or ameliorating RuV infection.

A panel of human antibody fragments were displayed on recombinant phage and RuV-specific binding activities have been detected in the phage library. The DNA sequences that encode the heavy and light chain variable regions of two antibody fragments and that have RuV-neutralizing activity were identified and named as DDF-RuV1 and DDF-RuV2. The corresponding protein sequences and the Complementarity Determining Regions (CDRs) that are responsible for the RuV-specific biological activity were determined. The binding activity of these Fabs can be further tested using novel libraries of peptides that are displayed on recombinant phage. Novel proteins are defined on the basis of the percentage of identity with isolated CDRs and variable regions for DDF-RuV1 and DDF-RuV2.

The nucleic acids of the invention can be used for producing recombinant proteins having RuV-specific binding and neutralizing properties, in the form of full antibodies, antibody fragments, bioactive peptides, or any other format of functional protein (in particular fusion proteins) using appropriate technologies and recombinant phage, prokaryotic host cells, or eukaryotic host cells.

The proteins of the invention can be used for treating detecting, treating, inhibiting, preventing, and/or ameliorating RuV infection. In particular, compositions having therapeutic, prophylactic, and/or diagnostic utility in the management of RuV infection can be prepared using these proteins, given their RuV-specific binding and neutralizing properties. These compositions may be used to supplement or replace RuV treatments that are based on antiviral compounds or intravenous immunoglobulin (IVIg) preparations, and can be suitable for ocular or topical administration.

Further embodiments of the present invention, including isolated DNA and protein sequences, vectors, recombinant phage, and host cells, as well as medical methods, compositions, and uses are provided in the following description.

### DESCRIPTION OF THE FIGURES

FIG. 1: Specificity of the RuV binding activity for preparations of recombinant phage expressing DDF-RuV1 and DDF-RuV2 or an unrelated human Fab (e137) that was used as a negative control. The binding activity was measured in ELISA using the indicated antigens for plate coating. (A) A total protein extract from a fibroblastic cell line (VERO cells) infected with a clinical isolate of RuV was used for plate coating. The two columns for each Fab refer to two different experiments. (B) Protein extracts from the fibroblastic cell line infected with a clinical isolate of RuV, from uninfected cells, or an unspecific purified protein (Bovine Serum Albumin, BSA) were used for plate coating.

FIG. 2: (A) Alignment of the DNA (lower case) and protein (upper case) sequence of the variable region for the heavy chain of DDF-RuV1 (DDF-RuV1 VH; SEQ ID NO.: 1 and 2). The predicted CDRs (DDF-RuV1 HCDR1, HCDR2, and HCDR3; SEQ ID NO.: 3, 4, and 5) are underlined. (B) Alignment of the DNA (lower case) and protein (upper case) sequence of the variable region for the light chain of DDF-RuV1 (DDF-RuV1 VL; SEQ ID NO.: 6 and 7). The predicted CDRs (DDF-RuV1 LCDR1, LCDR2, and LCDR3) are underlined.

FIG. 3: (A) Alignment of the DNA (lower case) and protein (upper case) sequence for the heavy chain of DDF-RuV2 (DDF-RuV2 VH; SEQ ID NO.: 8 and 9). The predicted CDRs (DDF-RuV2 HCDR1, HCDR2, and HCDR3; SEQ ID NO.: 10, 11, and 12) are underlined. (B) Alignment of the DNA (lower case) and protein (upper case) sequence for the light chain of human Fab DDF-RuV2 (DDF-RuV2 HC; SEQ ID NO.: 13 and 14). The predicted CDRs of this light chain (DDF-RuV2 LCDR1, LCDR2, and LCDR3) are underlined.

FIG. 4: RuV neutralization activity for human Fabs DDF-RuV1 (A) and DDF-RuV2 (B) when expressed as partially purified human recombinant Fabs and displayed on phage coat proteins. The dose-response analysis was based on the number of RuV-infected cells as measured by immunofluorescence. The percentage values were calculated by comparing the data on RuV infected cells obtained using RuV pre-incubated without any Fab (negative control).

FIG. 5: (A) Protein sequence for the heavy chain of human Fab DDF-RuV1, as expressed using pDLac-RuV1-FLAGhis vector (DDF-RuV1 HCtag; SEQ ID NO.: 15). The variable region of this heavy chain as originally cloned (SEQ ID NO.: 2) is underlined. The PelB sequence is comprised between amino acids 1 and 22. Amino acids 147-252 correspond to amino acids 1-106 of human Ig gamma-1 chain C region (SwissProt Acc. No.: P01857). Amino acids 253-267 correspond to the FLAGhis sequence. (B) Protein sequence for the light chain of human Fab DDF-RuV1, as expressed using pDLac-RuV1-FLAGhis vector (DDF-RuV1 LC; SEQ ID NO.: 16). The variable region of this light chain as originally cloned (SEQ ID NO.: 7) is underlined. The PelB sequence is comprised between amino acid 1 and 22. Amino acids 135-240 correspond to amino acids 1-106 of Ig kappa chain C region (SwissProt Acc. No.: P01834). (C) Protein sequence for the heavy chain of human Fab DDF-RuV2, as expressed using pDLac-RuV2-FLAGhis vector (DDF-RuV2 HCtag; SEQ ID NO.: 17). The variable region of this heavy chain as originally cloned (SEQ ID NO.: 9) is underlined. The PelB sequence is comprised between amino acids 1 and 22. Amino acids 146-251 correspond to amino acids 1-106 of human Ig gamma-1 chain C region (SwissProt Acc. No.: P01857). Amino acids 252-266 correspond to the FLAGhis sequence. (D) Protein sequence for the light chain of human Fab DDF-RuV2, as expressed using pDLac-RuV2-FLAGhis vector (DDF-RuV2 LC; SEQ ID NO.: 18). The variable region of this light chain as originally cloned (SEQ ID NO.: 14) is underlined. The PelB sequence is comprised between amino acid 1 and 22. Amino acids 129-234 correspond to amino acids 1-106 of Ig kappa chain C region (SwissProt Acc. No.: P01834). The sequences were confirmed by sequencing the inserts with primers designed within the constant region of either heavy or light chain.

FIG. 6: (A) Sequence of the random 12-mer peptide-cp3 fusion library as provided by the producer in the instruction manual for the Ph.D.-12 Phage Display Peptide Library Kit (available in New England Biolabs web site at http://www.neb.com/nebecomm/products/productE8110.asp). The position of the relevant restriction sites in the M13 XhoI-Forward (SEQ ID NO.: 19) and M13 SpeI-reverse (SEQ ID NO.: 20) primers are indicated by an arrow. The position of the relevant restriction sites in the M13 KpnI-Forward (SEQ ID NO.: 21) and M13 EagI-reverse (SEQ ID NO.: 22) primers are underlined. (B) Coding sequence of Ph.D.-12 Library (PhD 12mer) when cloned within the pDDcXSmer-orcp3 library (modified from FIG. 3 in WO 07/007154; SEQ ID NO.: 25). The position of the sequences that are provided by the original pDDc phagemid (the PelB signal peptide, the DIS linker, and cp3* 5′ end), is indicated. The relevant restriction sites are underlined. A linker sequence of 14 amino acids is positioned between the peptide library and the by N-terminus of cp3*. (C) Coding sequence of Ph.D.-12 Library (PhD 12mer) when cloned within the pDDcKE12mer-orcp3 library (modified from FIG. 3 in WO 07/007,154; SEQ ID NO.: 26). The position of the sequences that are provided by the original pDDc phagemid (the PelB signal peptide, the DIS linker, and cp3* 5′ end), is indicated. The relevant restriction sites are underlined. A linker sequence of 18 amino acids is positioned between the peptide library and the by N-terminus of cp3*. The KpnI and EagI sites were introduced by digesting pDDc with XhoI and SpeI and inserting two annealed oligonucleotides including HAtag sequence, KpnI site, EagI site, and with single stranded 5′ ends compatible with XhoI and SpeI restriction sites (SEQ ID NO.: 23 and 24). The vector was also modified by PCR in order to eliminate an EagI site within the Zeocin marker gene that is cloned in the DD cassette.

FIG. 7: Schematic representation of pDDcXSmer-orcp3 library and generation of pDDcXSmer-orcp3/cp8 library (modified from FIG. 15 in WO 07/007154). The phagemid maps indicates the position of the coat proteins (cp3* and cp8*), the Amplicillin marker gene within the phagemid backbone (Amp') and the replication origins (ColE1 and F1(+)). The following elements are present in the DD cassette (cloned between the Bgl I/Sfi I sites that are used for generating the pDDcXSmer-orcp3/cp8 library): the Ph.D peptide library (Lib), the pLacZ promoter fused to PeLB sequence (LPe), the stuffer sequence (St) and the Zeocin marker gene (Zed). The position of relevant restriction sites are indicated with arrows. The pDDcKEl2mer-orcp3 library and the pDDcKE12mer-orcp3/cp8 library present additional Kpn I and Eag I restriction sites (see FIG. 6C).

FIG. 8: Digestion of clones randomly selected from pDDcXSmer-orcp3/cp8 library without any panning (A) pDDcXSmer-orcp3/cp8 library after three rounds of panning (B), and pDDcKE12mer-orcp3/cp8 library after three rounds of panning (C). Isolated bacterial clones were used for performing miniprep of phagemid DNA which was digested with Nhe I and SpeI. The reactions were loaded on a 1% agarose gel for separating and comparing the resulting fragments (NS) with the corresponding undigested DNA (C). The clones presenting 3.8 and 0.6 Kb fragments have the DD cassette oriented to cp3*. The clones presenting 2.6 and 1.8 Kb fragments have the DD cassette oriented to cp8*. The arrows are positioned according to a marker DNA.

FIG. 9: Examples of sequences from clones randomly chosen in the following library: pDDcXSmer-orcp3 (A; SEQ ID NO.: 27-34), pDDcKE12mer-orcp3 (B; SEQ ID NO.: 35-42). Amber stop codons (tag) are suppressed into XL1-Blue E. coli strain and code for an amino acid (Q). The sequences were confirmed by sequencing the inserts with a primers designed within cp3*.

FIG. 10: Anti-HAtag colony screening. Bacterial colonies obtained after three rounds of panning against an anti-HAtag antibody were transferred onto a membrane and lysate in alkaline condition. For detecting the expression of HAtag in the original colonies, the membrane was incubated with the murine anti-HAtag antibody (the antibody that was used for panning) and then with an anti-mouse IgG1 conjugated to horseradish peroxidase in PBS/0.1% Tween-20 (dilution 1:1000). After three washes with PBS/0.1% Tween-20 the membranes were subjected to enhanced chemiluminescence detection using the Supersignal West Pico chemiluminescent substrate (Pierce).

### DETAILED DESCRIPTION OF THE INVENTION

The pDD phagemid and the related methods described in WO 07/007154 allow the cloning, the expression, and the selection of protein sequences that are fused to either one or the other of two predefined phage coat proteins. This approach allows the selection of identification of protein sequences that can be differentially expressed or displayed on surface or recombinant phage, and consequently selected from a phage display library with different efficiency.

In the present case, a phage library was constructed in a pDD phagemid by cloning the variable regions of human heavy and light chain immunoglobulins. The library was panned against protein extracts of RuV-infected cells and selected clones were subsequently tested in a cell-based assay for determining the ones that present RuV neutralizing activities. The DNA sequence that encode the two most promising clones, named DDF-RuV1 and DDF-RuV2, were determined and then cloned in appropriate vectors for bacterial expression.

The present invention provides novel protein sequences that are capable of binding and neutralizing RuV and that comprise specific CDRs (Complementarity Determining Regions) identified in the Fabs DDF-RuV1 or of DDF-RuV2. In particular, each of the HCDR3s (CDR3 of the heavy chain variable region) of the invention (SEQ ID NO.: 5 and SEQ ID NO.: 12) characterizes the antigen-binding portion of DDF-RuV1 and DDF-RuV2, respectively.

The HCDR3 is considered as characterizing the antigen-binding portion of an antibody and, consequently, providing the biological activity (e.g. binding and neutralizing RuV). Even though, several or all CDRs of an antibody are generally required for obtaining a complete antigen-binding surface, HCDR3 is the CDR showing the highest differences between antibodies. In fact, the diversity of HCDR3 sequence and length is fundamental for determining the specificity for most antibodies (Xu J and Davies M, 2000; Barrios Y et al. 2004; Bond C et al., 2003).

Proteins containing a specific HCDR3 of the invention as RuV binding moiety can be generated by combining it (or not) with other CDRs from the same Fab in which such HCDR3 was identified within an antibody protein framework (Knappik A et al., 2000). Combinations of CDRs can be linked to each other in very short proteins that retain the original binding properties without disrupting the original binding activity, even within a protein framework unrelated to antibody structure (Kiss C et al., 2006; Smith J et al., 1995).

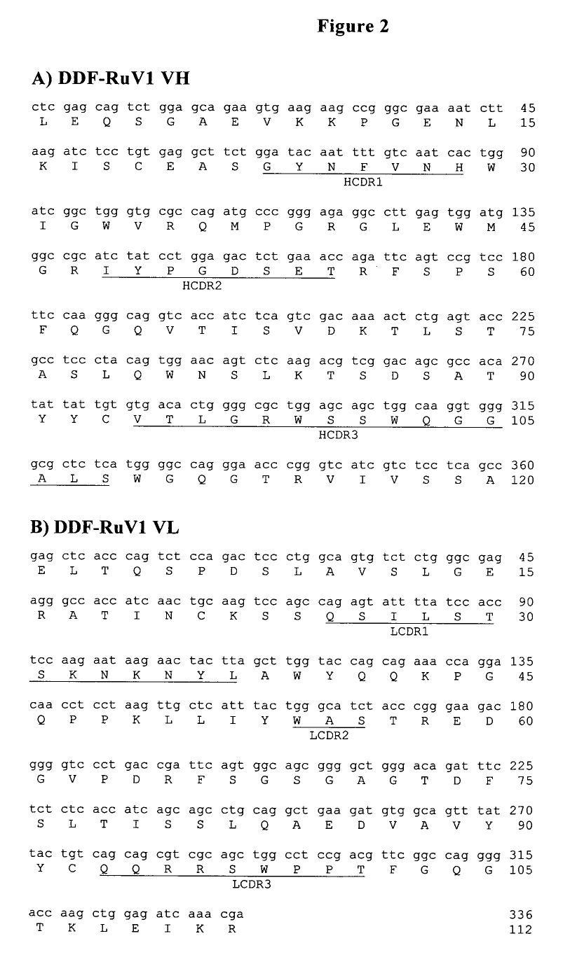

In one embodiment, the present invention provides a protein comprising a sequence having at least 90% identity with the HCDR3 of the Fab DDF-RuV1 (SEQ ID NO.: 5). Together with the HCDR1 and HCDR2, (SEQ ID NO.: 3 and SEQ ID NO.: 4; FIG. 2A), this HCDR3 is comprised in the variable region of the heavy chain of DDF-RuV1 Fab (DDF-RuV1 VH; SEQ ID NO.: 2). The variable region of a light chain that forms this Fab (DDF-RuV1 VL: SEQ ID NO.: 7), as well its specific LCDRs, (CDRs of the light chain variable region), have been determined (FIG. 2B).

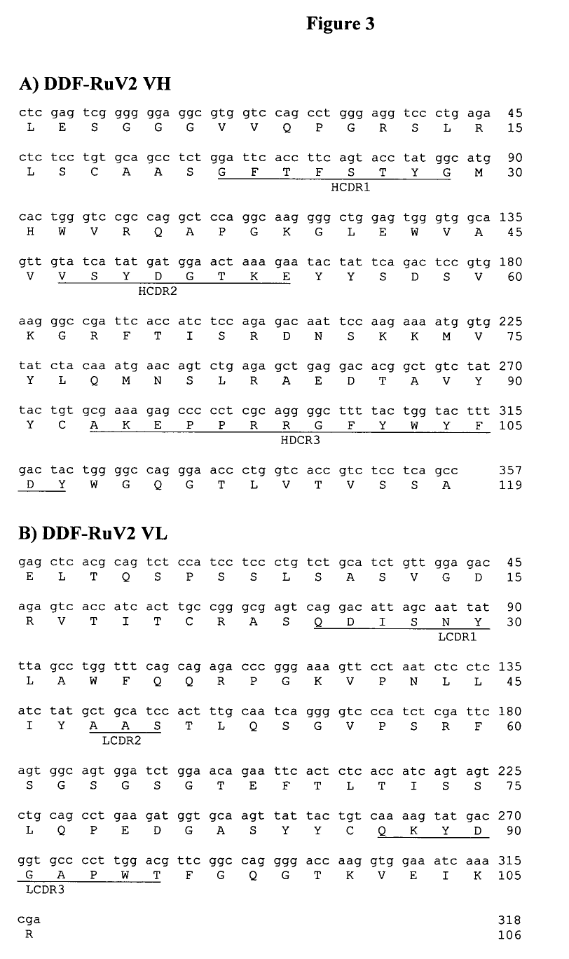

In another embodiment, the present invention provides protein comprising a sequence having at least 90% of identity with the HCDR3 of the Fab DDF-RuV2 (SEQ ID NO.: 12). Together with the HCDR1 and HCDR2 (SEQ ID NO.: 10 and SEQ ID NO.: 11; FIG. 3A), this HCDR3 is comprised in the variable region of the heavy chain of DDF-RuV2 (DDF-RuV2 VH; SEQ ID NO.: 9). The variable region of a light chain that forms this Fab (DDF-RuV1 VL; SEQ ID NO.: 14), as well its specific LCDRs, have been determined (FIG. 3B).

If the proteins of the Invention are based on the sequence of DDF-RuV1, they should comprise a sequence having at least 90% identity to SEQ ID NO.: 5. In particular, they should comprise a sequence having at least 90% identity with SEQ ID NO.: 2. More in particular, such proteins should also comprise one or more sequences selected from the group consisting of SEQ ID NO.:3 and SEQ ID NO.: 4. Proteins, in particular antibodies and antibodies fragments that are based on DDF-RuV1, can further comprises a sequence having at least 90% identity with SEQ ID NO.: 7.

Alternatively, if the proteins of the Invention are based on the sequence of DDF-RuV2, they should comprise a sequence having at least 90% identity to SEQ ID NO.: 12. In particular, they should comprise a sequence having at least 90% identity with SEQ ID NO.: 9. More in particular, such proteins should also comprise one or more sequences selected from the group consisting of SEQ ID NO.: 10 and SEQ ID NO.: 11. Proteins, in particular antibodies and antibodies fragments that are based on DDF-RuV2, can further comprises a sequence having at least 90% identity with SEQ ID NO.: 14.

Further embodiments of the Invention are the DNA sequences encoding the variable region of heavy and light chains of both Fabs, in particular those having at least 90% of identity with the original DNA sequences that have been cloned and determined for the variable regions of DDF-RuV1 (SEQ ID NO.: 1 for the heavy chain; SEQ ID NO.: 6 for the light chain) and DDF-RuV2 (SEQ ID NO.: 8 for the heavy chain; SEQ ID NO.:13 for the light chain).

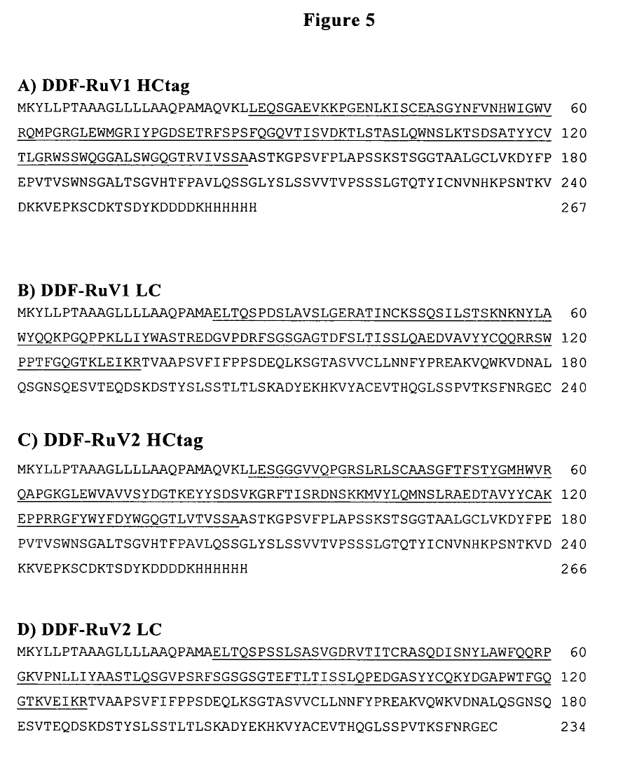

These DNA sequences (or selected portions, such as those encoding the isolated HCDRs and LCDRs as indicated in FIGS. 2 and 3) can be transferred in other vectors for expressing them within one of the known formats for recombinant antibodies (e.g. full, affinity-matured, CDR-grafted, or fragments as shown for tagged versions of DDF-RuV1 and DDF-RuV2 in FIG. 5), or fusion proteins to which they confer RuV binding and neutralizing properties.

Wherever a level of identity is indicated, this level of identity should be determined on the full length of the relevant sequence of the invention.

The variable region of the heavy and light chains forming either DDF-RuV1 or DDF-RuV2 (or selected portions, such as the isolated HCDRs and LCDRs) can be comprised within an antibody having a specific isotype, in particular within a fully human recombinant antibody. This antibody may comprise the VL and VH sequences of either DDF-RuV1 or DDF-RuV2 as light and heavy chains variable regions in the natural conformation of a tetrameric complex formed by two light and two heavy chains. When a fully human antibody is desirable, the antibody should further comprise a heavy chain constant region selected from the group consisting of human IgG1, IgG2, IgG3, IgG4, IgM, IgA and IgE constant regions. The IgG isotype, for example, is the antibody format of almost all approved therapeutic antibodies (Laffly E and Sodoyer R, 2005). However, antigen-binding portions isolated from a human IgG1 can be transferred on a human IgA sequence and the resulting recombinant antibody maintained the activity of the original IgG1, as recently shown with an antibody capable of inhibiting HIV infection (Mantis N et al., 2007).

Alternatively, the variable region of the heavy and light chains forming either DDF-RuV1 or DDF-RuV2 (or selected portions, such as the isolated HCDRs and LCDRs) can be comprised in any other protein format for functional antibody fragments, as described in the literature under different names such as Scfv (single-chain fragment variable), Fab (variable heavy/light chain heterodimer), diabody, peptabody, VHH (variable domain of heavy chain antibody), isolated heavy or light chains, bispecific antibodies, and other engineered antibody variants for non-/clinical applications (Jain M et al., 2007; Laffly E and Sodoyer R, 2005). For example, recombinant variants of DDF-RuV1 or DDF-RuV2 can be cloned in the pDD-compatible expression vector called pDLac-FLAGhis (PCT/IB2008/000266) and expressed in the form of tagged Fabs (SEQ ID NO.: 15-16, FIG. 5A-B for DDF-RuV1; SEQ ID NO.: 17-18, FIG. 5C-D for DDF-RuV2).

Alternative antibodies and antibody fragments can be generated using the sequences of DDF-RuV1 or DDF-RuV2 through a process for shuffling light chains. In fact, different antibodies and antibody fragments can be generated and tested for RuV-specific activity using a single heavy-chain variable domain VH (such as the one of either DDF-RuV1 or DDF-RuV2) that is combined with a library of VL sequences, for example using common phage display technologies or those described in WO 07/007154. This approach may allow determining VH/VL combinations with improved properties in terms of affinity, stability, specificity, and/or recombinant production (Rojas G et al., 2004; Suzuki K et al., 2007).

Moreover, it is known that antibodies may be modified in specific positions in order to have antibodies with improved features, in particular for clinical applications (such as better pharmacokinetic profile or higher affinity for an antigen). These changes can be made in the CDRs and/or framework of either DDF-RuV1 or DDF-RuV2. The sequence can be determined by applying any of the dedicated technologies for the rational design of antibodies that make use of affinity maturation and other methods (Kim S et al., 2005; Jain M et al., 2007).

Antibody-based strategies for developing new bioactive peptides also showed the feasibility of synthesizing CDR-derived peptides that contain L-amino acids and/or D-amino acids. These alternative molecules can maintain the original activity with a more appropriate pharmacological profile (Levi M et al., 2000; Wijkhuisen A et al., 2003). Thus, each of the novel HCDR3s, as well as sequences highly similar to them, fusion proteins containing them, and synthetic peptides derived from them (e.g. containing D-amino acids or in the retro-inverso conformation), can be tested and used as RuV-binding proteins.

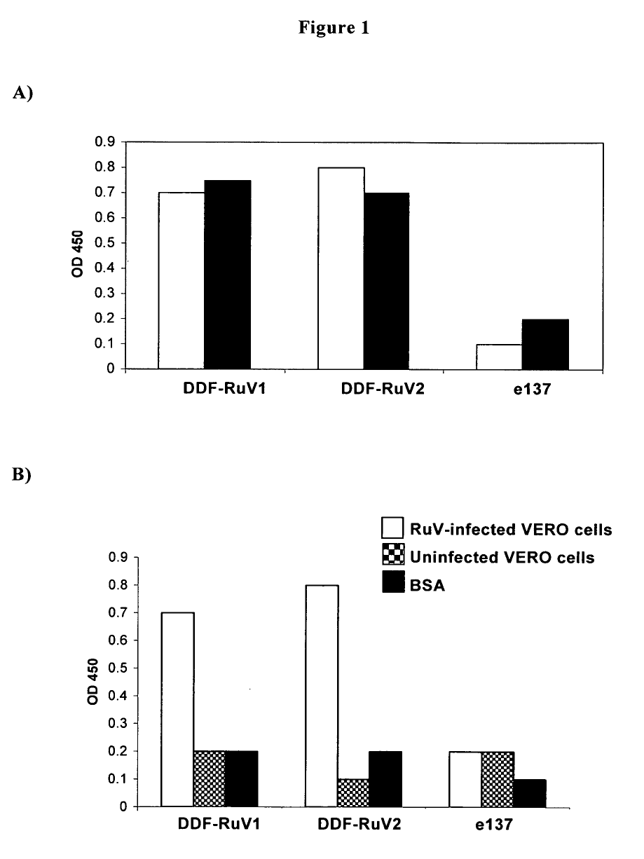

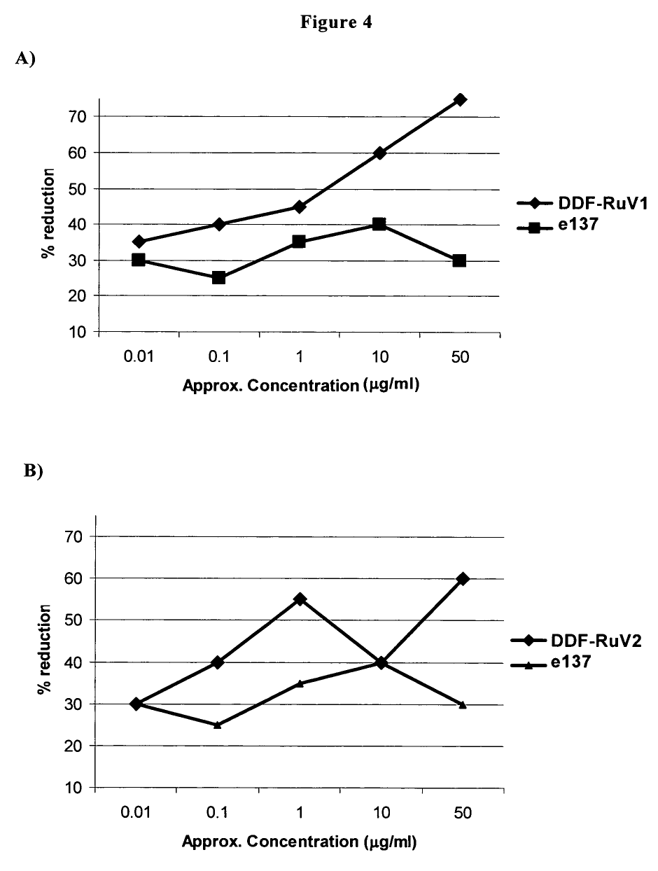

The proteins of the Invention can be also used for characterizing neutralizing antigens on RuV virion. In fact, DDF-RuV1 and DDF-RuV2 have been initially cloned due to their specific binding to cell extracts derived from a RuV-infected cell line in ELISA (FIG. 1) and their capability to neutralize RuV infection was then determined by an in vitro neutralization assay using a RuV clinical strain (FIG. 4). Consequently, the protein of the invention can be used for defining other RuV-binding proteins (in form of the full antibodies, antibody fragments, bioactive peptides, or fusion proteins) that are capable of neutralizing RuV infection and that compete with the protein of the invention, as determined by any relevant binding assay (e.g. ELISA that are described in the Examples). Such competing proteins may simply contain any of the HCDR3 sequences defined above, optionally together with HCDRs and LCDRs that are possibly in part or completely different from those identified in DDF-RuV1 or DDF-RuV2 sequences.

The proteins of the invention are provided as antibodies, antibody fragments, bioactive peptides, or fusion proteins that binds and neutralize RuV. These alternative proteins should maintain, if not enhance such properties as determined for DDF-RuV1 and DDF-RuV2 Fabs.

In the specific case of fusion proteins, the heterologous sequence(s) should be located in N- or C-terminal position to the RuV-specific binding and neutralizing moiety (e.g. the specific HCDR3 or variable region of an antibody fragment), without affecting negatively the correct expression and biological activity of such moiety.

The term “heterologous protein” indicates that a protein sequence is not naturally present in the N- or C-terminal position to the RuV-specific binding and neutralizing moiety (e.g. an antibody fragment). The DNA sequence encoding this protein sequence is generally fused by recombinant DNA technologies and comprises a sequence encoding at least 5 amino acids. This heterologous sequence is generally chosen for providing additional properties related to specific diagnostic and/or therapeutic uses. Examples of such additional properties include: better means for detection or purification, additional binding moieties or biological ligands, or the post-translational modification of the fusion protein (e.g. phosphorylation, glycosylation, ubiquitination, SUMOylation, or endoproteolytic cleavage).

Means for choosing and designing protein moieties, ligands, and appropriate linkers, as well as methods and strategies for the construction, purification, detection and use of fusion proteins are provided in the literature (Nilsson J et al., 1997; “Applications Of Chimeric Genes And Hybrid Proteins” Methods Enzymol. Vol. 326-328, Academic Press, 2000; WO 01/77137) and are commonly available in clinical and research laboratories. For example, the fusion protein may contain sequences recognized by commercial antibodies (including tags such as polyhistidine, FLAG, c-Myc, or HA tags; FIG. 5) that can facilitate the in vivo and/or in vitro identification of the fusion protein, or its purification. Other protein sequences can be easily identified by direct fluorescence analysis (as in the case of Green Fluorescent Protein), or by specific substrates or enzymes (using proteolytic sites, for example).

The therapeutic activity may be improved by the fusion with another therapeutic protein, such as another antiviral protein or a protein altering cell metabolism and/or activity. The stability of the RuV-specific antibodies, antibody fragments, and fusion proteins may be improved with the fusion with well-known carrier proteins, such as a phage coat protein (cp3 or cp8), Maltose Binding Protein (MBP), Bovine Serum Albumin (BSA), or Glutathione-S-Transferase (GST).

Alternatively (or additionally to the fusion to a heterologous protein sequence), the activity of the RuV-specific protein of the invention may be improved with the conjugation to different compound such as therapeutic or diagnostic agents. Examples of these agents are detectable labels (e.g. a radioisotope, a fluorescent compound, a colloidal metal, a chemiluminescent compound, a bioluminescent compound, or an enzyme) that can be bound using chemical linkers or polymers. The RuV-specific biological activity of a protein of the invention may be also improved by the fusion with a compound, such as a polymer altering the metabolism and/or the stability in diagnostic or therapeutic applications.

The proteins of the Invention (e.g. antibodies, antibody fragments, bioactive peptides, fusion proteins, or their conjugated variants) can be screened and characterized in order to demonstrate their capability to compete with the original DDF-RuV1 and DDf-RuV2, and possibly their similar or even superior capability of neutralizing RuV infection, as determined by any relevant assay described in the Examples or the literature.

The literature also provides several examples of technologies using which the RuV antigen and the specific epitope that is recognized by each Fab can be determined and compared to those determined in the past. For example, ELISA, immunoprecipitation, or Western Blot using RuV proteins, and related truncated variants or synthetic peptides, have used to determine relevant epitopes within E1 or E2 protein. In particular, antibodies, antibodies fragments, and the other proteins of the Invention can be tested in assays used for characterizing the RuV-specific biological activity and epitope for antibody fragments (Williamson R et al., 1993), murine or human monoclonal antibodies (Green K and Dorsett P, 1986; Cordoba P et al., 1997; Chaye H et al., 1992; Qiu Z et al., 1994; Ou D et al., 1992; Steenbakkers P et al., 1992; Hilfenhaus J et al., 1986; WO 07/011,698; WO 93/14206; WO 95/09232), and human sera (Giessauf A et al., 2004; Starkey W et al., 1995; Zrein M et al., 1993; Mitchell L et al., 1992). More extensive characterization and validation for RuV-related prophylactic, diagnostic, and therapeutic uses of the protein of the Invention can then be performed using one or more of the in vitro or in vivo assays (tissue- or cell-based assays, infection models established in rodents) that are disclosed in the literature for studying RuV pathogenesis and immunobiology, as summarized in the Background of the Invention (Cusi M et al., 1995; Duncan R et al., 1999; Cordoba P et al., 2000; Lee and Bowden, 2000; Garbutt M et al., 1999; Ou D et al., 1992; Tzeng W et al., 2005; Perrenoud G et al., 2004; Wang Z et al., 2003).

Further objects of the inventions are the nucleic acids encoding any of the antibodies, antibody fragments, fusion proteins or isolated CDRs defined above. The examples provide such sequences in particular as encoding the full variable regions of DDF-RuV1 and DDF-RuV2 (SEQ ID NO.: 1, 6, 8, and 13). The nucleic acid should have at least 90% identity with SEQ ID NO.:1, SEQ ID NO.:6, SEQ ID NO.:8 and/or SEQ ID NO.:13.

Such sequences, in particular those within them that are associated to specific CDRs (FIGS. 2 and 3), can be comprised in vectors and expression cassettes operably linked to a promoter, or cloned in a pDD-based phagemid. This specific type of vectors allows their expression as fusion proteins with either the cp3 or cp8 phage coat protein, and consequently the production of recombinant phage comprising such phagemid and expressing the Fabs of the invention on their surface. Thus, the recombinant phage comprising a phagemid vector that expresses a protein of the Invention can be used as a means for detecting and/or neutralizing RuV infection.

The pDD-based phagemids in which the sequences of the invention have been cloned and characterized by means of the corresponding recombinant phage, contain DNA sequences that can be transferred (in part or totally) into vectors where the original Fabs, or protein sequences derived from them, can be appropriately expressed as recombinant proteins in host cells (as shown in the Examples with the pDLac-FLAGhis system).

In the case of immunoglobulin variable chains (in particular human immunoglobulin variable chains) that are isolated using phage display technologies, an important modification is the conversion of the selected Fab or scFV into a full immunoglobulin protein having a preferred isotype and constant region. Thus, when a fully human antibody is desirable, the expression vector should further comprise a heavy chain constant region selected from the group consisting of IgG1, IgG2, IgG3, IgG4, IgM, IgA and IgE constant regions. This kind of modification allows, for example, generating full human monoclonal antibodies of all isotypes constructed from phage display library-derived single-chain Fv or Fabs and expressing in mammalian or insect cells. As widely described in the literature (Persic L et al., 1997; Guttieri M et al., 2003), vectors are specifically designed for expressing antibodies, allowing the fusion of this sequence to constant (Fc) regions of the desired isotype (for example, human IgG gamma1).

The antibodies or fusion proteins can be expressed as recombinant proteins in prokaryotic organisms (e.g. Escherichia coli; Sorensen H and Mortensen K, 2005; Venturi M et al., 2002), plants (Ma J et al., 2005), or eukaryotic cells, that allow a high level of expression as transient or stable transformed cells (Dinnis D and James D, 2005). This would be required in particular when the characterization of the antibodies has to be performed using more demanding functional and/or in vivo assays.

The literature provides different strategies for expressing a protein as a Fab or a similar format for antibody fragments, in prokaryotic host cells, as reviewed in articles and chapters of books (“Phage display: A Practical Approach”, vol. 266, ed. Clackson and Lowman H, Oxford Univ. Press, 2004; “Phage Display: A Laboratory Manual”, ed. Burton D et al., CSHL Press, 2001; Corisdeo S and Wang B, 2004; Benhar I, 2001).

When the protein, especially an antibody, is expressed in eukayotic host cells (mammalian cell lines, in particular), different vector and expression systems have been designed for generating stable pools of transfected cell lines (Aldrich T et al., 2003; Bianchi A and McGrew J, 2003). High level, optimized, stable expression of recombinant antibodies has been achieved (Schlatter S et al., 2005), also due to optimization of cell culture conditions (Grunberg J et al., 2003; Yoon S et al., 2004) and by selecting or engineering clones with higher levels of antibody production and secretion (Bohm E et al., 2004; Butler M, 2005).

The nucleic acid sequences encoding the sequence of interest (e.g. the relevant variable regions of the heavy and light chain) should be appropriately cloned in the expression cassette of a vector or of distinct vectors where they are operably linked to appropriate regulatory sequences (e.g. promoters, transcription terminators). The expression cassette should include a promoter, a ribosome binding site (if needed), the start codon, and the leader/secretion sequence, that can drive accordingly the expression of a mono or bicistronic transcript having the DNA coding for the desired protein.

The vectors should allow the expression of the protein of the Invention in the prokaryotic or eukaryotic host cell under the control of transcriptional initiation/termination regulatory sequences, which are chosen to be constitutively active or inducible. Methods for producing such proteins include culturing host cells transformed with the expression vectors comprising their coding sequences under conditions suitable for protein expression and recovering the protein from the host cell culture. The host cells comprising the nucleic acids of the invention can be prokaryotic or eukaryotic host cells and may allow the secretion of the desired recombinant protein. A cell line substantially enriched in such cells can be then isolated to provide a stable cell line.

These nucleic acids, recombinant phage, and host cells can be generated and used for producing a protein of the Invention by applying common recombinant DNA technologies. Briefly, the desired DNA sequences can be either extracted by digesting the phagemid with restriction enzymes, or amplified using the original phagemid as a template for a Polymerase Chain Reaction (PCR) and the PCR primers for specifically amplifying full variable regions of the heavy and light chains or only portions of them (such as HCDR3). Such DNA fragments can be then transferred into more appropriate vectors for further modification and/or expression into prokaryotic or eukaryotic host cells, as described in many books and reviews on how to clone and produce recombinant proteins, including some titles in the series “A Practical Approach” published by Oxford University Press (“DNA Cloning 2: Expression Systems”, 1995; “DNA Cloning 4: Mammalian Systems”, 1996; “Protein Expression”, 1999; “Protein Purification Techniques”, 2001).

The DNA sequence coding for the displayed and selected protein sequence, once inserted into a suitable episomal or non-homologously or homologously integrating vector, can be introduced in the appropriate host cells by any suitable means (transformation, transfection, conjugation, protoplast fusion, electroporation, calcium phosphate precipitation, direct microinjection, etc.). Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

For eukaryotic hosts (e.g. yeasts, insect or mammalian cells), different transcriptional and translational regulatory sequences may be employed, depending on the nature of the host. They may be derived from viral sources, such as adenovirus, bovine Papilloma virus, Simian virus or the like, where the regulatory signals are associated with a particular gene which has a high level of expression. Examples are the TK promoter of the Herpes virus, the SV40 early promoter, the yeast gal4 gene promoter, etc. Transcriptional initiation regulatory signals may be selected which allow for the transient (or constitutive) repression and activation and, consequently, for modulating gene expression. During further cloning steps, the sequence encoding the antibody or the fusion protein can be adapted and recloned in other vectors for specific modifications at the DNA level only at both the DNA and protein level. These changes can be determined, for example, using software for selecting the DNA sequence in which the codon usage and the restriction sites are the most appropriate for cloning and expressing a recombinant protein using specific vectors and host cells (Rodi D et al., 2002; Grote A et al., 2005; Carton J et al., 2007). Protein sequences can also be added in connection to the desired antibody format (Scfv, Fab, fully human antibody, etc.), or to the insertion, substitution, or elimination of one or more internal amino acids.

These technologies can also be used for further structural and functional characterization and optimization of the therapeutic properties of antibodies (Kim S et al., 2005), or for generating vectors allowing their stable in vivo delivery (Fang J et al., 2005). For example, recombinant antibodies can also be modified at the level of structure and/or activity by choosing a specific Fc region to be fused to the variable regions (Furebring C et al., 2002; Logtenberg T, 2007), by generating recombinant single chain antibody fragments (Gilliland L et al., 1996), by fusing stabilizing peptide sequences (WO 01/49713), or by adding radiochemicals or polymers to chemically modified residues (Chapman A et al., 1999).

The cells which have been stably transformed by the introduced DNA can be selected by also introducing one or more markers which allow for selection of host cells which contain the expression vector. The marker may also provide for phototrophy to an auxotropic host, biocide resistance, e.g. antibiotics, or heavy metals such as copper, or the like, and it may be cleavable or repressed if needed. The selectable marker gene can either be directly linked to the DNA gene sequences to be expressed, or introduced into the same cell by co-transfection. Additional transcriptional regulatory elements may also be needed for optimal expression.

Host cells may be either prokaryotic or eukaryotic. Amongst prokaryotic host cells, the preferred ones are B. subtilis and E. coli. Amongst eukaryotic host cells, the preferred ones are yeast, insect cells (using baculovirus-based expression systems), or mammalian cells, such as human, monkey, mouse, insect (using baculovirus-based expression systems) and Chinese Hamster Ovary (CHO) cells, because they provide post-translational modifications to protein molecules, including correct folding or certain forms of glycosylation at correct sites. Also yeast cells can carry out post-translational peptide modifications including glycosylation. A number of recombinant DNA strategies exist which utilize strong promoter sequences and high copy number of plasmids that can be utilized for production of the desired proteins in yeast. Yeast recognize leader sequences in cloned mammalian gene products and secrete peptides bearing leader sequences (i.e., pre-peptides).

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may proliferate using tissue culture techniques appropriate to the cell type. A cell line substantially enriched in such cells can be then isolated to provide a stable cell line. The host cells can be further selected on the basis of the expression level of the recombinant protein.

The antibody, the antibody fragments, the bioactive peptides, the fusion proteins, and any other compound defined above as being capable of binding and neutralizing RuV can be provided using the well-established technologies that allow purifying them as recombinant proteins from cell culture or from synthetic preparations (i.e. any conventional procedure involving extraction, precipitation, chromatography, electrophoresis, or the like). These preparations should provide a sufficient amount of recombinant protein (from the microgram to the milligram range) to perform a more extensive characterization and validation for RuV-related prophylactic, diagnostic, and therapeutic uses.

Methods for antibody purification can make use of immobilized gel matrix contained within a column (Nisnevitch M and Firer M, 2001; Huse K et al., 2002; Horenstein A et al., 2003) and in particular on the general affinity of antibodies for substrates such protein A, protein G, or synthetic substrates (Verdoliva A et al., 2002; Roque A et al., 2004), as well as by antigen- or epitope-based affinity chromatography (Murray A et al., 2002; Jensen L et al., 2004). After washing, the protein is eluted from the gel by a change in pH or ionic strength. Alternatively, HPLC (High Performance Liquid Chromatography) can be used. The elution can be carried out using a water-acetonitrile-based solvent commonly employed for protein purification.

The preparations of recombinant proteins can be tested in in vitro or in vivo assays (biochemical, tissue- or cell-based assays, disease models established in rodents, biophysical methods for affinity measurements, epitope mapping, etc.), in particular using one or more of those disclosed in the literature for studying RuV pathogenesis and immunobiology, for example using wild-type and reference RuV strains as defined according to the International health conventions (WHO 2005; WHO 2006).

The antibody, the antibody fragments, the bioactive peptides, the fusion proteins, and any other compound defined above as being capable of binding and neutralizing RuV can be used for detecting, treating, inhibiting, preventing, and/or ameliorating RuV infection. To this purpose, such compounds can be used for preparing diagnostic, therapeutic, or prophylactic compositions for the medical management of RuV infection.

These compositions may comprise an antibody, antibody fragment, fusion proteins, bioactive peptides, and any other compound defined above on the basis of the sequence and activity of human DDF-RuV1 and DDF-RuV2. The compositions may further comprise a different RuV-neutralizing antibody or antibody fragment, an intravenous immunoglobulins (IVIg) preparation, and/or an antiviral compound. The different RuV-neutralizing antibody or antibody fragment should be characterized by a different neutralizing epitope, such as the ones already described in the literature. In fact, the literature shows many examples in which, when two or more antibodies directed to a viral or human target are combined in a pharmaceutical composition, the resulting composition may have an improved therapeutic efficacy due not to a simple additive effect but to a specific synergic effect (Logtenberg T, 2007).

Pharmaceutical compositions may optionally comprise any pharmaceutically acceptable vehicle or carrier. These compositions may further comprise (or may be administered together with) any additional therapeutic or prophylactic agent, such as vaccines, intravenous immunoglobulin preparations, or antiviral compounds. Recent literature also suggests that human monoclonal antibodies can be used for supplementing (and replacing, if possible) present intravenous immunoglobulin preparations, giving the opportunity to reduce frequency and/or dosage of such pharmaceutical compositions (Bayry J et al., 2007).

The compositions that comprise any of the proteins (e.g. antibodies, antibody fragments, fusion proteins, bioactive peptides) and of the nucleic acids defined above can be administered to an individual with a RuV-related diagnostic, therapeutic, or prophylactic purpose. These compositions can be administered as means for RuV-specific passive immunization which provide therapeutic compounds (in particular therapeutic antibodies or therapeutic antibodies fragments) that, by targeting RuV virions, can inhibit the propagation of the virus in the treated patient, and potentially block the outbreak of a viral infection in the population.

Depending on the specific use, the composition should provide the compound to the human subject (being an infant, a pregnant woman, an elderly individual, or any other individual that is infected by RuV or considered at risk for RuV due to the contact with a RuV-infected individual, to hospitalization, or to immunosuppressive/chemotherapeutic treatments) for a longer or shorter period of time. To this purpose, the composition can be administered intramuscularly, intravenously, subcutaneously, topically, mucosally, by a nebulizer, an inhaler, as eyedrops in non-/biodegradable matrix materials, or using particulate drug delivery systems such as microbeads.

A pharmaceutical composition should provide a therapeutically or prophylactically effective amount of the compound to the subject that allows the compound to exert its activity for a sufficient period of time, in particular for ocular or topical administration, given the presence of the virus in the eye and in cutaneous rash associated to secondary viremia. Compositions comprising antibodies or antibody fragments proved to be effective when applied topically to wounds, skin, mucosae, or cornea (Brereton H et al., 2005; Castle P et al., 2002; Streit M et al., 2006; Nwanegbo E et al., 2007).

The desired effect is to improve the status of the patient by controlling RuV infection, reactivation, and/or re-infection, and by reducing at least some of the clinical manifestations of RuV infection. For example, the composition should be administered at an effective amount from about 0.005 to about 50 mg/kg/body weight, depending on the route of administration and the status of the individual.

In the case of composition having diagnostic uses, the compound should be detected using technologies commonly established in the clinical and research laboratories for detecting virus in biological samples (e.g. ELISA or other serological assays), or, when administered to a subject in vivo, at least 1, 2, 5, 10, 24, or more hours after administration. The detection of RuV can be performed, using the proteins of the invention, in substitution or coupled to the known means and procedures that have been established for monitoring RuV infection in at risk populations of both immunocompetent and immunocompromised hosts.

A method for treatment, prophylaxis, or diagnosis of RuV, or of RuV-related disease can comprise the administration of a protein or of a nucleic acid as above defined. The method may further comprise the administration of a different RuV-neutralizing antibody or antibody fragment, an intravenous immunoglobulins (IVIg) preparation, and/or an antiviral compound.

The clinical development and use should be based on the characterization of the antibody pharmacokinetics and pharmacodynamics (Lobo E et al., 2004), the preclinical and clinical safety data (Tabrizi M and Riskos L, 2007), and compliancy official requirements for commercial manufacturing scale formulation and analytical characterization of therapeutic recombinant antibodies for therapeutic and in vivo diagnostic use in humans (Harris R et al. 2004).

The invention will now be described by means of the following Examples, which should not be construed as in any way limiting the present invention.

### EXAMPLES

### Example 1

### Expression and Selection of Human Fabs that bind RuV Protein Extracts in ELISA

Materials & Methods

Library Construction

The cDNA encoding for heavy and light chains of human IgG1 was obtained from lymphocytes obtained from a RuV-seropositive individual according to the literature (Burioni et al., 1998; “Phage Display: A laboratory Manual”, Burton D R et al., CSHL Press, 2001). The phage library was constructed using a cloning cassette compatible with a pDD vector according to the technology described in the PCT patent application WO07/007154. The Fabs were cloned within a DD cassette and expressed on the surface of the recombinant phage in the library. The selection of human Fabs through the panning of the pDD-based Fab library and the sequencing of the positive clones was performed as described in the literature (Burioni R et al. 1998).

The specific CDRs of the Fabs were defined by comparing the predictions and sequence alignments provided by IMGT/V-QUEST (Giudicelli V et al., 2004) and other databases containing protein sequences of human antibodies, such as those provided by the European Bioinformatics Institute and searchable using FASTA (http://www.ebi.ac.uk/fasta33/index.html).

Preparation of Protein Extracts from Cells in Cell Culture

The VERO cell line (ATCC Acc. No. CCL-81) is a simian fibroblastic cell line that grows as a monolayer and commonly used for testing and propagating human viruses, including RuV. Neutralizing antibodies against RuV have been characterized using VERO cells (Cordoba P et al., 2000b).

VERO cells that have been infected with a RuV clinical isolate (H2), were used for the preparation of the RuV-specific material for panning the phage display library and testing the Fabs in ELISA. VERO cells are maintained in Modified Eagle Medium containing 10% of foetal bovine serum inactivated (FBS), 50 μg/ml of penicillin, 100 μg/ml streptomycine and 2 mM L-glutamine.

The cells (RuV-infected or uninfected VERO) were scraped and resuspended in 250 ml of lysis buffer (50 mM Tris-HCl pH 8.0; 150 mM NaCl; 0.02% Sodium Azide; 0.5% Triton-X), incubated for 20 minutes on ice, then centrifuged 12000 rpm for 2 minutes at 4° C. The protein concentration of the resulting supernatants was determined in duplicates using BCA Protein Assay kit (Pierce) and reported with reference to a serial dilution of Bovine Serum Albumin (BSA) at known concentrations (0 mg/ml=Blank−2.000 mg/ml=max. value). The absorbance of all the samples was measured with the spectrophotometer set to 540 nm.

Panning and ELISA Using Protein Extracts

The protein coating for ELISA was performed on 96-well plates using the following antigens: lysate of VERO cells, infected or not infected with RuV (clinical isolate H2), Bovine Serum Albumin (BSA). Each sample was diluted in carbonate buffer (100 nanograms of total protein in 25 μl final volume per well) and the plate was incubated overnight at 4° C. After washing with distilled water, the plate was blocked by incubation in PBS with 1% BSA for 1 hour at 37° C.

The ELISA was performed using 40 l of undiluted sample containing the following Fabs using a protocol disclosed in the literature (Bugli F et al., 2001): DDF-RuV1, DDF-RuV2, and e137, an unrelated Fab specific for Hepatitis C virus. The Fabs were tested in duplicate wells. After incubation with each Fab for 1 hour at 37° C. and five washings with PBS with 0.1% Tween-20, 40 μl goat anti-human Fab, peroxidase-conjugate (Sigma; Cat no. A0293) were added and incubated for 1 hour at 37° C. Plate washing was repeated as above and enzymatic reaction was developed by adding 40 μl of substrate (TMB Substrate Kit; Pierce) to each well. ELISA reactions were developed for 15 minutes at 37° C. Enzyme activity was stopped by adding stop solution (H₂SO₄) and the absorbance measured with a spectrophotometer set to 450 nanometers.

Fab Preparation for ELISA and Neutralization Assay

The protocol was similar to those described in the literature using pDD or other phagemids (“Molecular Cloning: A Laboratory Manual”, Sambrook et al., Cold Spring Harbor Press, NY, 1989; Burioni R et al., 1998; Bugli F et al., 2001; WO 07/007154).

Briefly, individual E. coli clones of the library were grown in 200 ml Super Broth (SB; 3.5% bacto-tryptone, 2% yeast extract, 0.5% NaCl) medium with antibiotics and supplemented with IPTG, harvested and washed with phosphate-buffered saline (PBS). Lysis was limited to the periplasmic space by sonication at 4° C. with controlled pulses. Fabs in the periplasmic extracts were partially purified by ultracentrifugation at 12,000 rpm in a JA-10 rotor for 45 minutes at 4° C. Product was filtered and concentrated 10 times with Centricon filters.

The concentration of the partially purified Fabs was determined in the periplasmic extracts by sandwich ELISA using ImmunoPure Goat Anti-Human IgG F(ab')₂ (Pierce; Cat. No. 31132), which was bound onto the surface of 96-well plate (Costar; Cat no. 3690). After a 1-hour incubation at 37° C., the plate was washed 6 times with deionized water and blocked using 170 μl/well PBS with 3% BSA. After a further 1-hour incubation at 37° C., 50 μl of a serial 3-fold dilution of each Fab, or known concentrations of a control human Fab (Cappel Labs; Cat. No. 6001-0100), in PBS with 1% BSA were added to each well and incubated at 37° C. for 1 hour. The plate was then washed 6 times with TPBS (PBS with 0.05% Tween-20). The antibody binding was then determined by adding 50 μl of alkaline phosphatase conjugated goat anti-human antibody (Pierce; Cat. No. 31312) and incubated at 37° C. for 1 hour. Plate washing was repeated as above with TPBS and 100 μl disodium p-nitrophenyl phosphate (Sigma) was added to each well. ELISA reactions were developed for 60 minutes and the results were plotted against the control human Fab diluted at the known concentrations. The absorbance of all the samples was measured with the spectrophotometer set to 590 nm.

Results

A library of recombinant phage was generated according to the pDD technology (WO07/007154) and panned on protein extracts obtained from a fibroblastic cell line infected with a clinical isolate of RuV. The five rounds of panning were also performed in parallel with the same library using a control protein extract from the same cell line not infected with RuV.

By the third round, the phage titre of the sample panned against the control protein extract was below 10⁴, meanwhile the phage titre of the sample panned against the RuV extract was more than 10⁴ at the fourth round, reaching 10⁵ at the fifth round. This value demonstrates the progressive enrichment of the library in recombinant phage expressing on their surface Fabs that bind RuV antigens.

Fourteen clones obtained after the fifth round of panning were individually tested in ELISA and confirmed as positive. The PCR and sequence analysis of the HCDR3 in the selected clones identified two heavy chains characterizing the human Fabs now named DDF-RuV1 and DDF-RuV2. The reactivity of recombinant phage expressing DDF-RuV1 or DDF-RuV2 was tested against the RuV-specific protein extract as well as against an unrelated antigens (Bovine Serum Albumin, protein extract from uninfected cells), confirming their binding activity in ELISA format (FIG. 1). The specificity of the binding was also confirmed using a control human Fab.

The DNA sequences of the full heavy and light chains variable regions of these Fabs were determined, together with the corresponding CDRs, for DDF-RuV1 and DDF-RuV2 (FIGS. 2 and 3, respectively). These Fabs were recloned also into other vectors for obtaining sufficient recombinant protein for further assays, using E. coli-based systems for protein expression.

### Example 2

### Properties of DDF-RuV1 and DDF-RuV2 Tested on RuV-Infected Cell Cultures

Materials & Methods

Neutralization Assay of RuV-Specific Human Fabs

The immunofluorescence-based assay was performed in Costar 24-well plates using 10⁴-10⁵ VERO cells, inoculated into the plates under conditions where confluent monolayers usually form after 72 hours incubation at 37° C. The RuV clinical isolate H2 was used for the infection.

The Fabs were partially purified as indicated in Example 1 and mixed at various concentrations (0.01, 0.1, 1, 10 and 50 μg/ml) with equal volumes of RuV cell-free stock at 50 TCID₅₀ (50% tissue culture infective dose) suspended in maintenance medium. The controls were constituted of equal volumes of maintenance medium and virus, in the absence of Fabs (blank control), or in the presence of an irrelevant Fab specific for human Hepatitis C virus (e137; Bugli F et al., 2001).

After 1 hour of incubation at 37° C., 250 μl of virus-fragment Fab mixtures or control mixtures were inoculated into wells (in duplicate) from which medium was removed. The plates were incubated for 2 hours at 37° C. to allow adsorption of unneutralized virus. The inocula were removed and 1.5 ml of maintenance medium was added. After 1 week of incubation in cell culture conditions, cells were washed with PBS, fixed with cold methanol-acetone solution (1:2 ratio) for 10 minutes at room temperature. Fixed cells were incubated with a commercial murine monoclonal antibody specific for protein E1 of RuV (Chemicon; Cat. No. MAB925) for 30 minutes at 37° C. in a humid atmosphere, washed with PBS and finally incubated with anti-mouse IgG FITC-conjugate (Sigma) for 30 minutes at 37° C. in humid atmosphere. The slides were washed with PBS, counterstained with Evans Blue dye and mounted with glycerol. Neutralizing ability of each Fab was determined by counting single fluorescing cells that are observed by fluorescence microscope (Olympus), and calculating the percentage of reduction in number of RuV-positive cells compared with the cells in the control samples (without Fab preincubation).

Results

The analysis of the RuV-neutralizing activity for both DDF-RuV1 and DDF-RuV2 was performed by using preparations of partially purified Fabs and an immunofluorescence-based assay.

The data on the reduction of RuV-infected cells indicate that DDF-RuV1 and DDF-RuV2 are endowed with a neutralizing activity when preincubated with RuV. In fact, when compared to the controls (RuV without a Fab or with an unrelated Fab), the addition of these Fabs reduce the number of infected cells in a dose-dependent manner (FIG. 4).

The experimental evidence presented here makes DDF-RuV1 and DDF-RuV2 Fabs (or alternative protein sequences based on their specific HCDR3s and showing similar properties) candidate compounds for diagnostic, therapeutic, or prophylactic applications related to RuV infection.

DDF-RuV1 and DDF-RuV2 can be expressed as recombinant proteins in bacterial host cells. In particular, these Fabs can be expressed by recloning their coding sequence in a vector compatible with the restriction sites present in a DD cassette such as a pDLac-FLAGhis vector (PCT/IB2008/000266). This vector allows the expression of soluble Fabs that contain appropriate constant regions that form the heterodimeric complex, and both a 6-His and Flag tag at the C-terminus of the heavy chain (FIG. 5). This approach allows a more direct cloning, detection (e.g. in ELISA) and purification (e.g. using a procedure based on immobilized metal affinity column, easier and less expensive compared to the immuno-affinity purification) of the Fabs.

### Example 3

### Construction and Validation of pDD-Based Peptide Libraries Suitable for Determining RuV-Specific Neutralizing Epitopes

The RuV-neutralizing Fabs have been identified initially by panning a pDD-based library of human Fabs using protein extracts of VERO cells that have been infected with RuV for the panning. Then the RuV-specific neutralizing activity was tested in a cell-based model, but without defining the viral epitope that is actually by DDF-RuV1 and/or DDF-RuV2.

Similar epitopes have been determined using different approaches in the past, including synthetic peptide ELISA (Giessauf A et al., 2004), protein deletion mutants (Chaye H et al., 1992), competition analysis using panels of murine monoclonal antibodies (Green K and Dorsett P, 1986), or combinations of these techniques (Wolinsky J et al., 1993).

At this scope, the pDD Technology can be applied for constructing peptide libraries that are displayed on phage surface on either one or the other of two functional phage coat proteins, as exemplified by making use of hemagglutinin epitope tag (HAtag) in WO 07/007154. Libraries of non-random or random peptides displayed using pDD Technology can be screened to select phage displaying peptides that specifically bind antibodies. The peptide sequence motifs that are selected in the library allow the definition of antibody-specific epitopes (Zhong G et al., 1997; Yip Y et al., 1999a).

The importance of screening and comparing peptide sequences that are displayed using different phage display formats has been demonstrated in the literature. In a first example, the epitope of a monoclonal antibody has been selected within phage-displayed epitopes (or “phagotope”) using two phage display libraries each containing peptides of a different length and fused to different phage coat proteins. The outcome of the screening was that different peptide sequences were selected using the two phage display platforms, suggesting that the characterization of phagotopes can require the comparison types of peptide presentation in phage coat proteins (O′Connor K et al., 2005). Similar evidences were found also in the case of peptides that bind organic (e.g. human hair or skin) or inorganic (e.g. polypropylene) supports. By screening and comparing commercial phage-displayed libraries of peptides having different length, as such or as modified according to a method described in the literature (Scholle M et al., 2005), the resulting sequences differ considerably (WO 07/035531, WO 06/094093, WO 06/094094, WO 06/094095).

Two distinct peptide libraries were constructed by combining a pDD phagemid (pDDc) with a commercial library (Ph.D.12 Phage Display Library Kit; New England BioLabs, cat, no. E8110S) that have been already used in the past for determining linear or conformational epitopes of antibodies (Li Y et al., 2007; Petit M et al., 2003) and modified for generating peptide libraries of different length (WO 07/035,531, WO 06/094093, WO 06/094094, WO 06/094095). This library contains only 32 of 64 possible codons since the third position is limited to T or G, increasing the relative frequency of residues with a single codon, as well as removing 2 of the 3 stop codons (the third one can be suppressed and translated into Glutamine in the appropriate E. coli strain).

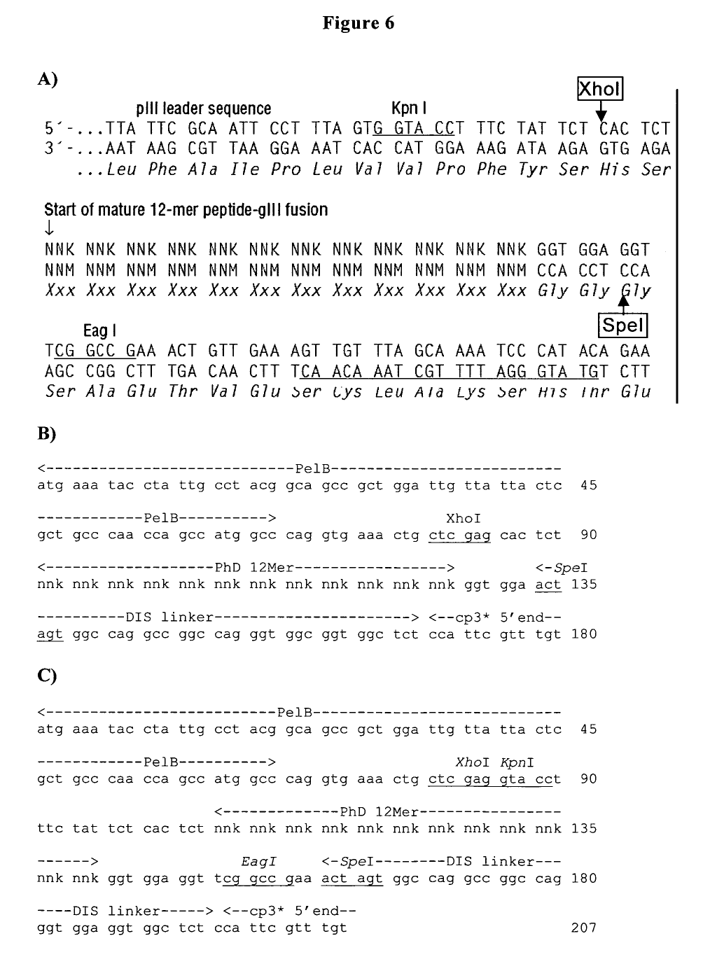

The replicative, double stranded form of the phagemids in this library was used as template for PCR amplification using a forward primer that contains a XhoI restriction site and a reverse primer that contains a SpeI restriction site. A second PCR amplification was performed using the same library as template and a forward primer that contains a KpnI restriction site and a reverse primer that contains an EagI restriction site (FIG. 6A).

The DNA fragments resulting from the PCR amplifications were digested and cloned into the pDDc vector for generating two distinct pDD-based peptide libraries, each having a title of approx. 10⁸ and initially fused only to cp3. In the first case, the pDDcXSmer-orcp3 library was generated by cloning the PCR amplification products in the XhoI and SpeI restriction sites (FIG. 6B). In the second case, the pDDcKE12mer-orcp3 library was generated by cloning the PCR amplification in the intermediate pDDc KE vector in which the KpnI and EagI restriction sites between the XhoI/SpeI sites and the 5′/3′ ends of the HA coding region (FIG. 6C; WO 07/007154).

It is important to note that the use of SpeI as cloning site provides a specific effect since this restriction enzyme cuts within a sequence (ACTAGT) that corresponds to two consecutive codons present in the original peptide library: ACT, coding for Threonine, and AGT, coding for Serine. Since the other two possible frames are not allowed within the library (i.e. A and C are not present as third position in the codons), it means that making use of SpeI for cloning the library within a DD cassette will allow the introduction of shorter variants of the peptides with a low frequency if the SpeI site in the pDD vector has the same coding frame, as in the case of pDDc (FIGS. 6B and 6C).

Such an approach may be used for generating variants of this type of library making use of other enzymes having recognition sites with similar properties (A or C as first base, A or C as second base, G or T as third base, recognizing 6 or 9 base pairs) such as A1wNI (recognizing CAGNNNCTG), NdeI (recognizing CATATG), HindIII (recognizing AAGCTT), or PvuII (recognizing CAGCTG). Similar sites may be also used to randomly integrate additional non-/random codons in the original library, further improving the complexity of the library.

The two pDD-based peptide libraries, in order to display the peptides fused to cp3 or cp8, need to be maintained and amplified (for example in the XL1-blue E. coli strain). The phagemids are purified and digested with BglI (to extract the DD cassette), and then religated to a BglI-digested pDDc vector. The ligation reaction is used for transforming bacteria so that the two libraries can be selected and maintained as Zeocin-resistant bacterial cells where the phagemids can amplified, included in recombinant phage following the infection with an helper phage, and single clones can be analyzed following the desired screening.

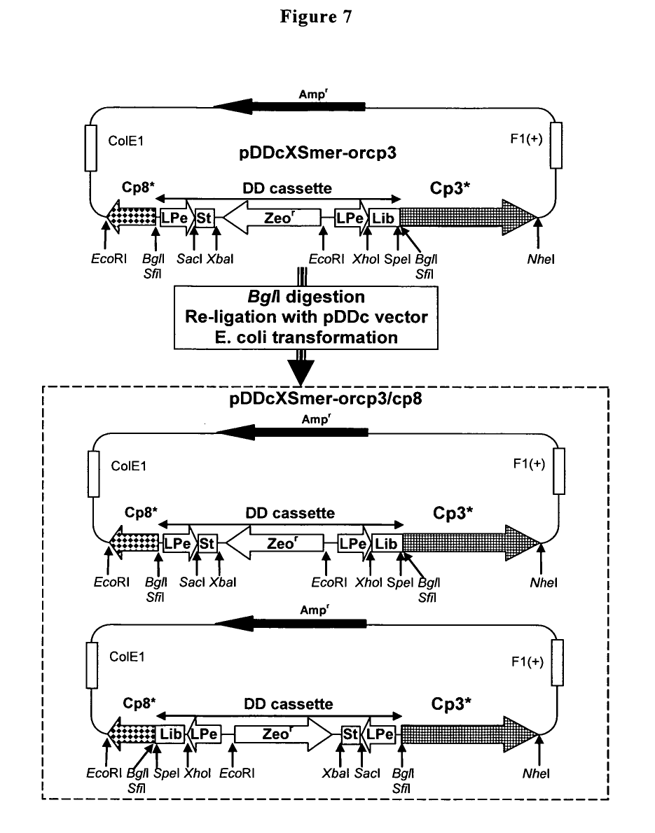

In this manner the peptide library in the DD cassette is cloned and screened in either one of two possible orientations in the corresponding pDDcXSmer-orcp3/cp8 and pDDcKE12mer-orcp3/cp8 libraries (FIG. 7; WO 07/007154). The libraries were then amplified in E. coli and recombinant phage were tested by comparing the outcome of the panning experiments. Panning involves the use of helper phage for generating the recombinant phage. This population of phage can be isolated and tested against a target on the basis of its binding affinity to a target of interest coated onto a microwell plate, as described in the literature. Following three to five consecutive panning rounds against the target of interest, a specific binding is manifested by an increase in the number of eluted phage when compared to a control.

In the present case, the two libraries were tested against an antibody having a well known linear epitope, such as a commercial anti-HAtag antibody (mouse monoclonal antibody HA.11, clonel6B12; Covance). This approach has been used previously for evaluating techniques for improving phage display platforms (Stratmann T and Kang A, 2005; Vanhercke T et al., 2005).

Even though there is reduction in the title of the library (due to the procedure involving the BglI digestion, religation, and E. coli transformation), the comparison of the panning against the relevant target (Anti-HAtag monoclonal antibody) or a negative control (BSA) show that, for both pDDcXSmer-orcp3/cp8 (Table I) and pDDcKE12mer-orcp3/cp8 (Table II) libraries there is a significant difference for the calculated input and output value. Moreover, the third round of panning shows that a specific enrichment of 10² for the output of anti-HAtag compared to the one of BSA.

The orientation in which the DD cassette and the peptide is present in each clone of the two libraries can be easily determined by a restriction analysis (FIG. 7). The digestion is performed by combining an enzyme cutting near the end of the DD cassette with the sequence to be displayed (such as XhoI or SpeI) and an enzyme cutting at the end of the sequence coding a coat protein in the backbone of the pDD vector (e.g. NheI for cp3*). The resulting restriction pattern will present a DNA fragment including only the sequence coding the coat protein (if the DD cassette is oriented toward cp3*) or a DNA fragment including the DD cassette and the sequence coding the coat protein (if the DD cassette is oriented toward cp8*).

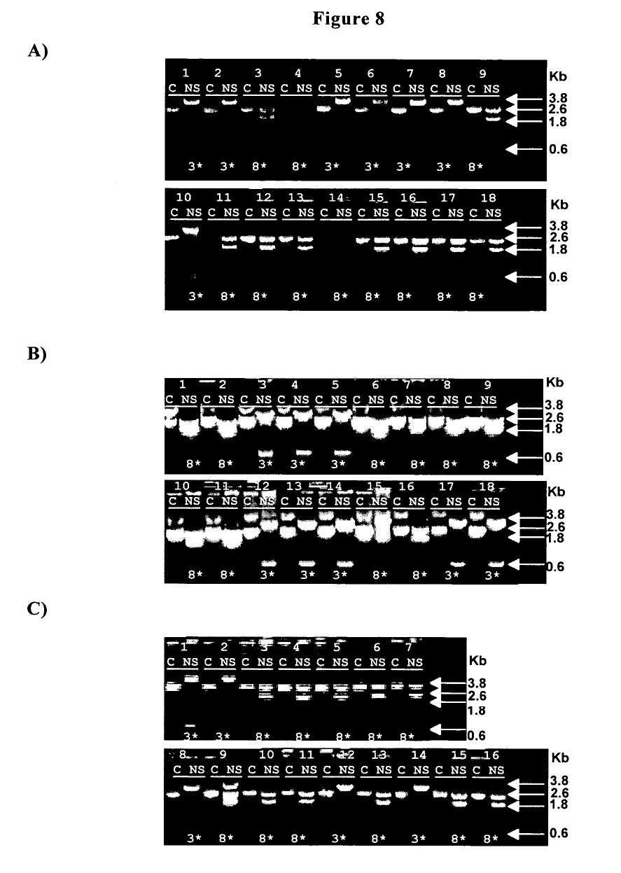

The NheI/SpeI digestion has been performed in panels of clones randomly chosen from the two libraries, before and/or after the panning described above. When the DNA fragments resulting from the digestion are separated by electrophoresis, a fragment of approx. 0.6 kb is detected when the DD cassette is oriented in cp3* and a fragment of approx. 1.8 kb is detected when the DD cassette is cp8* oriented. This analysis showed a slight prevalence of clones having a cp8* orientation in all the situations (FIG. 8).

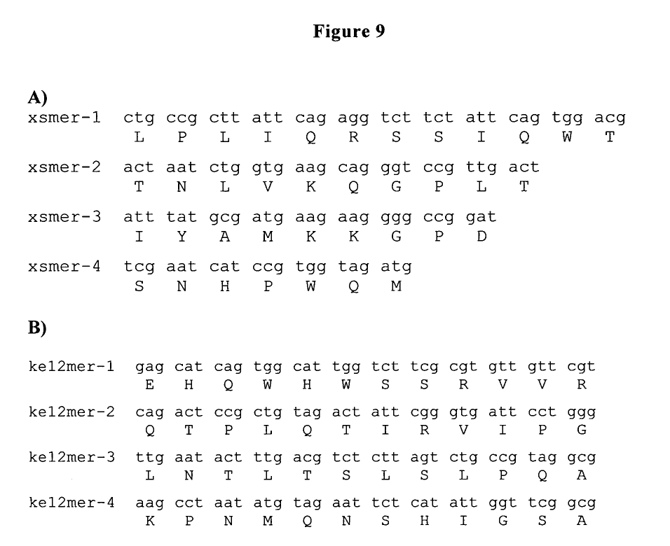

Sequences of peptides cloned in the pDDcXSmer-orcp3/cp8 and pDDcKE12mer-orcp3/cp8 libraries were determined for clones that were randomly chosen, confirming both the good level of variability and, in the case of pDDcXSmer-orcp3/cp8, the presence of peptides having shorter sequences, ranging from 7 to 12 amino acids (FIG. 9). The same analysis was made in clones obtained after panning pDDcKE12mer-orcp3/cp8 against anti-HAtag antibody and the presence of peptides comprising the core sequence of HAtag (the tetrapeptide YPYD) was confirmed in selected clones.

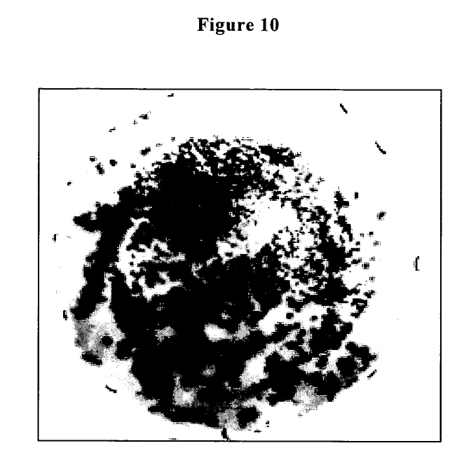

As a further confirmation of the presence and the selection of clones coding HAtag specific peptide in the libraries, bacterial clones generated after panning selection were transferred onto a nitrocellulose-like membrane and lysate in alkaline condition. In this way the bacterial proteins are immobilised on the nitrocellulose paper, which is then incubated with anti-HAtag. A large amount of clones presenting the HAtag is confirmed (FIG. 10).

The present strategy for generating and panning pDD-based peptide libraries where bioactive peptides (e.g. epitopes that characterize antibodies, inhibitors or recognition sites of proteases, peptides that binding membrane receptors, signaling proteins, or transcription factors) are identified, can be adapted for the different uses or cloning strategies. Conditions for improving and evaluating the complexity of a random peptide library displayed on phage are described in the literature (Noren K et al., 2001; Rodi D et al., 2002).

For instance, the original peptide library (when it is desirable to maintain its length and complexity) can be cloned using different combinations of restriction enzymes satisfying a combination of criteria which may allow a better efficiency of the cloning and transformation process, For example, the restriction enzymes BstEII, MluI, Agel, EcoRV, SalI, SnaBI, and BglII do not cut neither into pDDc vector and the Ph.D 12 library, and (compared to EagI) are methylation insensitive.

This example provides pDD-based peptide library comprising a plurality of fusion proteins, each fusion protein comprising a viral surface protein (cp3* or cp8*) and a random peptide sequence that are linked by a sequence comprising a DD linker in a pDD vector. The structural, functional, and sequence features of pDD vectors and DD cassettes are defined in WO 07/007154.

The initial peptide library should comprise codons having general formula NNK (N is any base, K is G or T). Following the digestion with a restriction enzyme that recognize sites compatible with the codon usage and the frame in the pDD vector as defined above, the library will be actually cloned as full or shorter variants of the original sequence. These peptide variants can be displayed on the outer surface of a genetic package in general, and in particular of a bacterial cell or a filamentous phage.

A method of constructing a library of phagemids for displaying peptides having different lengths and fused to either one or the other of two functional phage coat proteins within the DD cassette of pDD vector comprises:
a) Digesting a DNA library (e.g. Ph.D 12 peptide library) that includes at least a combination of codons corresponding to a restriction site (e.g. SpeI) to be used for cloning said library into a DD cassette with at least said restriction enzyme;
b) Ligating the resulting DNA library to a phagemid (e.g. pDDc) digested with at least the same restriction enzyme;
c) Using this first library of phagemids for transforming bacterial cells and generating a first library of bacterial cells;
d) Isolating the phagemids from said first library of bacterial cells;
e) Digesting said phagemids with a restriction enzyme present in the DD linker of the of DD cassette;
f) Ligating the resulting DD cassette to a pDD vector digested with the same restriction enzyme;
g) Using this second library of phagemids for transforming bacterial cells and generating a second library of bacterial cells;
h) Isolating the phagemids from said first library of bacterial cells.

At the end of this process, the pDD-based peptide library results operationally linked to the regulatory sequences in the DD cassette and to the sequences of either one or the other functional phage coat proteins in the pDD vector (e.g. cp3* or cp8*). The library of bacterial cells containing such phagemids can be infected with helper phage in order to obtain the population of recombinant phage that are used for the panning or any other type of negative/positive selection.

This cloning approach combines the fusion of either one of two phage coat proteins (as indicated for pDD Technology in WO 07/007154) to the modification of the length of the coded peptide. This method provides novel libraries of phagemids encoding peptides and novel libraries of peptides that can be screened against the desired target. In fact, a plurality of phagemids obtainable according to this method can be used for transforming E. coli. The resulting cells can be then infected with a helper phage for generating the library of recombinant phage.

The pDD-based peptide libraries can be included (as phagemids or as bacterial cells) in a kit further comprising primers for amplifying and/or sequencing the DNA sequence coding specific peptides that are selected from the original population of phage-displayed random.

The pDD-based peptide libraries can be used for generating recombinant phage that can be screened for selecting peptides having a particular biological activity or to isolate and purify peptides that bind a specific ligand molecule. In the case of in vitro panning, such libraries can be used for epitope mapping (O′Connor K et al., 2005; Zhong G et al., 1997; Yip Y et al., 1999a), for mapping protease specificity and designing protease inhibitors (Diamond S, 2007; Sedlacek R and Chen E, 2005) or screening antigens on cell surfaces (Mutuberria et al., 2004). In the case of in vivo panning, such libraries (and in particular those like pDDcXSmer-orcp3/cp8 library that display the peptides not only fused to either cp3 or cp8, but also having the different length) allow to identify protein-ligand interactions in extracellular space and on the cell membrane (Finger A et al., 2002; Yip Y et al., 1999b).

TABLE I

| | Transformation (Titre of the Library) | | | |
|---|---|---|---|---|
| | 100 ng of I pDDcXSmer-orcp3/cp8 and 100 μl of | | | |
| | electrocompetent XL1Blue E. coli (10⁵) | | | |
| | Coating Conditions for Panning | | | |
| | anti-HAtag antibody | | BSA | |
| | (300 ng/well in 50 μl PBS | | (50 μl/well | |
| | pH 8.6) | | PBS + BSA1%) | |
| I ROUND | INPUT | 6 × 10⁸ | INPUT | 4 × 10⁸ |
| (5 wells) | (cfu/ul) | | (cfu/ul) | |
| | OUTPUT | 10⁷ | OUTPUT | 8 × 10⁶ |
| | (cfu/ul) | | (cfu/ul) | |
| II ROUND | INPUT | 5.5 × 10⁸ | INPUT | 5 × 10⁸ |
| (5 wells) | (cfu/ul) | | (cfu/ul) | |
| | OUTPUT | 2 × 10⁶ | OUTPUT | 2.5 × 10⁵ |
| | (cfu/ul) | | (cfu/ul) | |
| III ROUND | INPUT | 6.5 × 10⁸ | INPUT | 4 × 10⁸ |
| (4 wells) | (cfu/ul) | | (cfu/ul) | |
| | OUTPUT | 2 × 10⁷ | OUTPUT | 3.5 × 10⁵ |
| | (cfu/ul) | | (cfu/ul) | |

TABLE II

| | Transformation (Titre of the Library) | | | |
|---|---|---|---|---|
| | 100 ng pDDcKE12mer-orcp3/cp8 and 100 μl of | | | |
| | electrocompetent XL1Blue E. coli (10⁶) | | | |
| | Coating Conditions for Panning | | | |
| | anti-HAtag antibody | | BSA | |
| | (300 ng/well in | | (50 μl/well | |
| | 50 μl PBS pH 8.6) | | PBS + BSA1%) | |
| I ROUND (5 wells) | INPUT | 4 × 10⁸ | INPUT | 4.5 × 10⁸ |
| | (cfu/ul) | | (cfu/ul) | |
| | OUTPUT | 6.9 × 0⁵ | OUTPUT | 7.5 × 10⁵ |
| | (cfu/ul) | | (cfu/ul) | |
| II ROUND (5 wells) | INPUT | 5.3 × 10⁸ | INPUT | 5 × 10⁸ |
| | (cfu/ul) | | (cfu/ul) | |
| | OUTPUT | 1.2 × 10⁶ | OUTPUT | 3.5 × 10⁵ |
| | (cfu/ul) | | (cfu/ul) | |
| III ROUND (4 wells) | INPUT | 6.2 × 10⁸ | INPUT | 3.2 × 10⁸ |
| | (cfu/ul) | | (cfu/ul) | |
| | OUTPUT | 2.6 × 10⁷ | OUTPUT | 3.6 × 10⁵ |
| | (cfu/ul) | | (cfu/ul) | |

### REFERENCES

Adamo Metal., (2008). Virology. 370:1-11.
Aldrich T et al., (2003). Biotechnol Prog. 19: 1433-8.
Am. Acad. Pediatr. Com. Infect. Dis., (2007). Pediatrics 120: 221-31.
Andrews J, (2004). Curr Opin Obstet. Gynecol. 16: 163-6.
Atreya C et al., (2004). Birth Defect Res A lin Mol. Terat. 70: 431-7.
Banatvala J, and Brown D (2004). Lancet. 363: 1127-37.
Banerji A et al., (2005). CMAJ. 172 : 1678-9.
Barrios Y et al., (2004). J Mol. Recognit. 17: 332-8.
Bayry J et al., (2007). Nat Clin Pract Neurology. 3: 120-1.
Bekker V et al., (2006). Pediatrics. 118: e315-22.
Benhar I (2001). Biotechol Adv. 19: 1-33.
Bianchi A and McGrew J (2003). Biotechnol Bioeng. 84: 439-44.
Bohm E et al., (2004). Biotechnol Bioeng. 88: 699-706.
Bond C et al., (2003). J Mol. Biol. 332: 643-55.
Bradbury A and Marks J (2004). J Immunol Methods. 290: 29-49.
Brereton H et al., (2005). Br J. Opthalmol. 89: 1205-9.
Burioni R et al., (1998). Hepatology. 28: 810-4.
Bugli F et al., (2001). J. Virol., 75: 9986-9990.
Butler M (2005). Appl Microbiol Biotechnol. 68: 283-91.
Carton J et al., (2007). Protein Expr Purif. 55: 279-86.
Castle P et al., (2002). J Reprod Immunol. 56: 61-76.
Chakravarti A and Jain M, (2006). 49: 54-6.
Chapman A et al., (1999). Nat. Biotechnol. 17: 780-3.
Chatenoud L, (2005). Methods Mol. Med. 109: 297-328.
Chaye H et al., (1992), Virology. 189: 483-92.
Claus C et al., (2006). J Gen Virol. 87: 3029-37.
Conrad U and Scheller J, (2005). Comb Chem High Thr Screen. 8: 117-26.
Cooray S et al., (2005). Virol J. 2:1.
Cordoba P et al., (1997). Viral Immunol. 10: 103-10.
Cordoba P et al., (2000a). Clin Diagn Lab Immunol. 7: 964-6.
Cordoba P et al., (2000b). Viral Immunol. 13: 83-92.
Corisdeo S, and Wang B, (2004) Protein Expr Purif. 34: 270-9.
Cusi M et al., (1995) Virus Res. 37: 199-208.
De Groot-Mijnes Jet al., (2006). Am J. Opthalmol. 141: 212-4.
Diamond S, (2007). Curr Opin Chem. Biol. 11: 46-51.
Dinnis D and James D, (2005). Biotechnol Bioeng. 91: 180-9.
Domegan L and Atkins G, (2002). J Gen Virol. 83: 2135-43.
Duncan R et al., (1999). Virology. 255: 117-28.
Dunman P and Nesin M, (2003). Curr Opin Pharmacol. 3: 486-96.
Fang J et al., (2005). Nat. Biotechnol. 23: 584-90.
Figuereido A et al., (2000). Rev Saude Publica. 34: 353-7.
Finger A et al., (2002). J Immunol Methods. 264: 173-186.
Furebring C et al., (2002). Mol. Immunol. 38: 833-40.
Garbutt M et al., (1999). J. Virol. 73: 3524-33.
Giessauf A et al., (2004). J Immunol Methods. 287: 1-11.
Giessauf A et al., (2005). Arch Virol. 150: 2077-90.
Gilliland L et al., (1996). Tissue Antigens. 47 : 1-20.
Giudicelli V et al., (2004). Nucl. Acids Res. 32: W435-440.
Green K and Dorsett P, (1986). J. Virol. 57 : 893-8.
Greenaway C et al., (2007). Ann Intern Med. 146: 20-4.
Grote A et al. (2005). Nucleic Acids Res. 33: W526-31.
Grunberg J et al., (2003). Biotechniques. 34 : 968-72.
Guttieri M et al., (2003). Hybrid Hybridomics 22: 135-45.
Harris R et al. (2004) Drug Development Research. 61 137-154.
Hilfenhaus Jet al., (1986). Behring Inst Mitt. 80: 31-41.
Hinman A et al., (2002). Bull World Health Organ. 80: 264-70.
Hofmann J et al., (1999). J Gen Virol. 80: 1657-64.
Hofmann J et al., (2000). Virus Res. 68: 155-60.
Horenstein A et al., (2003). J Immunol Methods. 275: 99-112.
Huse K et al., (2002). J Biochem Biophys Methods. 51: 217-31.
Hust M and Dubel S, (2005). Methods Mol Biol 295: 71-96.
Jain M et al., (2007). Trends Biotechnol. 25: 307-16.
Jensen L et al., (2004). J Immunol Methods. 284: 45-54.
Kawamura N et al., (2000). Eur J. Pediatr. 159: 764-6
Keller M and Stiehm E, (2000). Clin Microbiol Rev. 13: 602-14.
Ki M et al., (2002). Epidemiol Infect. 129: 557-64.
Kim S et al., (2005). Mol. Cells. 20: 17-29.
Kiss C et al., (2006). Nucleic Acids Res. 34: e132.
Knappik A et al., (2000). J Mol. Biol. 296: 57-86.
Krause I et al., (2002). Transfus Med. 12: 133-9.
Kremer J et al., (2007). Vaccine. 24: 2594-601.
Laffly E and Sodoyer R, (2005). Hum. Antibodies. 14: 33-55.
Lee J and Bowden D, (2000). Clin Microbiol Rev. 13: 571-587.
Lee Jet al., (1999). Virology. 265: 110-9.
Levi M et al., (2000). AIDS Res Hum Retroviruses. 16: 59-65.
Li Y et al., (2007). J Immunol Methods. 325: 1-8.
Lobo E et al., (2004). J Pharm Sci. 93: 2645-68.
Logtenberg T, (2007). Trends Biotechnol. 25: 390-4.
Lund K and Chantler J, (2000). J. Virol. 74: 796-804.
Ma J et al., (2005). Vaccine. 23: 1814-8.
Mancini N et al., (2004). New Microbiol. 27: 315-28.
Mantis Net al., (2007). J. Immunol. 179: 3144-52.
Masuko-Hongo K et al., (2003). Best Pract Res Clin Rheumatol. 17: 309-318
Matter L et al., (1997). J Infect Dis. 175: 749-55.
Mauracher C et al., (1992) J Med. Virol. 36: 202-8.
Mendelson E et al., (2006). Reprod Toxicol. 21: 350-82.
Mitchell L et al., (1992). J Clin Microbiol. 30: 1841-7.
Mitchell L et al., (1993). Virus Res. 29: 33-57.
Mugnaini C et al., (2007). Eur J Med. Chem. 42: 256-62.
Murray A et al., (2002). J Chromatogr Sci. 40: 343-9.
Mutuberria Ret al., (2004). J Immunol Methods. 287: 31-47.
Nicoara C et al., (1999). Clin Diagn Lab Immunol. 6: 868-871.
Nilsson J et al., (1997). Protein Expr Purif. 11: 1-16.
Nilsson A et al., (2002). Pediatrics. 109: e91.
Nisnevitch M and Firer M (2001), J Biochem Biophys Methods. 49: 467-80.
Noren K et al., (2001). Methods. 23: 169-78.
Nwanegbo E et al., (2007) Invest Opthalmol V is Sci. 48: 4171-6.
O′Connor K et al., (2005). J Immunol Methods. 299: 21-35.
Orellana A et al., (1999). J. Biotechnol. 75: 209-19.
Ou D et al., (1992). J. Virol. 1992. 66: 1674-81.
Pebody R et al., (2000). Epidemiol Infect. 125: 347-57.
Perrenoud G et al., (2004). Vaccine. 23: 480-8.
Persic L et al., (1997). Gene. 187: 9-18.
Petit M et al., (2003). J Biol. Chem. 278: 44385-92.
Qiu Z et al., (1994). J. Virol. 68: 4086-91.
Reef S et al., (2002). JAMA. 287: 464-472.
Robinson K et al., (1995). FEMS Immunol Med. Microbiol. 10: 191-8.
Rodi D et al. (2002). J Mol. Biol. 322: 1039-52.
Roque A et al., (2004). Biotechnol Prog, 20: 639-5.
Rota M et al., (2007). Epidemiol Infect. 135: 555-62.
Rojas G et al., (2004). J Immunol Methods. 293 : 71-83.
Schlatter S et al., (2005). Biotechnol Prog, 21: 122-33.
Scholle Met al., (2005). Comb Chem High Throughput Screen. 8: 545-51.
Sedlacek R and Chen E, (2005). Comb Chem High Throu Screen. 8: 197-203.
Semerikov V et al., (2000). Epidemiol Infect. 125: 359-66.
Smith J et al., (1995). J Biol. Chem. 270, 30486-490.
Sorensen H and Mortensen K, (2005). Microb Cell Fact. 4, 1.
Starkey W et al., (1995). J Clin Microbiol. 33: 270-4.
Steenbakkers P et al., (1992). J Immunol Methods. 152: 69-77.
Stratmann T and Kang A, (2005). Proteome Sci. 3: 7.
Streit M et al., (2006). Int Wound J. 3: 171-9.
Suzuki K et al., (2007). J Med. Virol. 79: 852-862.
Tabrizi M and Riskos L, (2007). Drug Disc Today. 12: 540-7.
Takahashi S et al., (1998). Clin Diagn Lab. 5: 24-7.
Terry G et al., (1988). Arch Virol. 98: 189-197.
Thomas H et al., (1993). J Med. Virol. 41: 196-200.
Tzeng W et al., (2005). J Clin Microbiol. 43: 879-85.
Ushida M et al., (2003) Jpn J Infect Dis. 56: 68-9.
Vanhercke T et al., (2005). J Biomol Screen. 10: 108-17.
Van Tilburg C et al., (2006). Leukemia. 20: 1717-22.
Venturi Metal., (2002). J Mol. Biol. 315: 1-8.
Verdoliva A et al., (2002). J Immunol Methods. 271: 77-8.
Wang Z et al., (2003). Intervirology. 46: 79-85.
WHO (2005) Wkly Epidemiol Rec. 80: 126-132.
WHO (2006) Wkly Epidemiol Rec. 81: 474-9.
Wijkhuisen A et al., (2003). Eur J. Pharmacol. 468: 175-82.
Williamson R et al., (1993). Proc Natl Acad. Sci. 90: 4141-5.
Wilson K et al. (2006). Clin Vaccine Immunol. 13: 380-6.
Wolinsky J et al., (1991). J. Virol. 65: 3986-94.
Wolinsky J et al., (1993). J. Virol. 67: 961-8.
Xu J and Davis M. (2000). Immunity. 13: 37-45.
Yip Y et al., (1999a). Comb Chem High Throughput Screen. 2: 125-138.
Yip Y et al., (1999b). J Immunol Methods. 225: 171-8.
Yoon S et al., (2004). Biotechnol Prog. 20: 1683-8.
Yu J et al., (2007). Pediatric Blood Canc. 49: 656-60.
Zhang T et al., (1992). J Clin Microbiol. 30: 824-30.
Zheng D et al., (2003). Emerg Infect Dis. 9: 1523-30.
Zhong G et al., (1997). J lnd Microbiol Biotechnol. 19: 71-6.
Zrein Metal., (1993). J Clin Microbiol. 31: 1521-4.

1. A protein comprising a sequence having at least 90% identity with SEQ ID NO.: 5.

2. The protein according to claim 1, wherein said protein comprises a sequence having at least 90% identity with SEQ ID NO.: 2.

3. The protein of claim 1, wherein said protein further comprises a sequence having at least 90% identity with SEQ ID NO.: 7.

4. The protein of claim 1, wherein said protein is an antibody, an antibody fragment, a bioactive peptide, or a fusion protein.

5. The protein of claim 4, wherein said antibody fragment is a variable heavy/light chain heterodimer, or a single-chain fragment variable.

6. The protein of claim 1 wherein such protein binds and neutralizes Rubella virus (RuV).

7. A nucleic acid molecule encoding a protein of claim 1.

8. The nucleic acid molecule of claim 7, wherein said nucleic acid has at least 90% identity with of SEQ ID NO.: 1.

9. A vector comprising a nucleic acid of claim 7.

10. A recombinant phage, a prokaryotic host cell, or an eukaryotic host cell comprising a nucleic acid of claim 7.

11. (canceled)

12. A method of detecting, treating, inhibiting, preventing, and/or ameliorating an RuV infection using a protein of claim 1.

13. A therapeutic, prophylactic, or diagnostic composition comprising a protein of claim 1.

14. The composition of claim 13 wherein the composition is for ocular or topical administration.

15. The composition of claim 13, further comprising a different RuV-neutralizing antibody, a different RuV-neutralizing antibody fragment, an intravenous immunoglobulins preparation, and/or an antiviral compound.

16. A method of producing a protein comprising a sequence having at least 90% identity with SEQ ID No.:5 using a nucleic acid of claim 7.

17. A method of producing a protein comprising a sequence having at least 90% identity with SEQ ID No.:5 using a host cell of claim 10.


SEQUENCE LISTING (42 sequences)

SEQ ID NO: 1 - DNA, 360 bp, Homo sapiens
ctcgagcagtctggagcagaagtgaagaagccgggcgaaaatcttaagatctcctgtgaggcttctggatacaattttgtcaatcactggatcggctgggtgcgccagatgcccgggagaggccttgagtggatgggccgcatctatcctggagactctgaaaccagattcagtccgtccttccaagggcaggtcaccatctcagtcgacaaaactctgagtaccgcctccctacagtggaacagtctcaagacgtcggacagcgccacatattattgtgtgacactggggcgctggagcagctggcaaggtggggcgctctcatggggccagggaacccgggtcatcgtctcctcagcc

SEQ ID NO: 2 - protein, 120 aa, Homo sapiens
LEQSGAEVKKPGENLKISCEASGYNFVNHWIGWVRQMPGRGLEWMGRIYPGDSETRFSPSFQGQVTISVDKTLSTASLQWNSLKTSDSATYYCVTLGRWSSWQGGALSWGQGTRVIVSSA

SEQ ID NO: 3 - protein, 7 aa, Homo sapiens
GYNFVNH

SEQ ID NO: 4 - protein, 8 aa, Homo sapiens
IYPGDSET

SEQ ID NO: 5 - protein, 15 aa, Homo sapiens
VTLGRWSSWQGGALS

SEQ ID NO: 6 - DNA, 336 bp, Homo sapiens
gagctcacccagtctccagactccctggcagtgtctctgggcgagagggccaccatcaactgcaagtccagccagagtattttatccacctccaagaataagaactacttagcttggtaccagcagaaaccaggacaacctcctaagttgctcatttactgggcatctacccgggaagacggggtccctgaccgattcagtggcagcggggctgggacagatttctctctcaccatcagcagcctgcaggctgaagatgtggcagtttattactgtcagcagcgtcgcagctggcctccgacgttcggccaggggaccaagctggagatcaaacga

SEQ ID NO: 7 - protein, 112 aa, Homo sapiens
ELTQSPDSLAVSLGERATINCKSSQSILSTSKNKNYLAWYQQKPGQPPKLLIYWASTREDGVPDRFSGSGAGTDFSLTISSLQAEDVAVYYCQQRRSWPPTFGQGTKLEIKR

SEQ ID NO: 8 - DNA, 357 bp, Homo sapiens
ctcgagtcggggggaggcgtggtccagcctgggaggtccctgagactctcctgtgcagcctctggattcaccttcagtacctatggcatgcactgggtccgccaggctccaggcaaggggctggagtgggtggcagttgtatcatatgatggaactaaagaatactattcagactccgtgaagggccgattcaccatctccagagacaattccaagaaaatggtgtatctacaaatgaacagtctgagagctgaggacacggctgtctattactgtgcgaaagagccccctcgcaggggcttttactggtactttgactactggggccagggaaccctggtcaccgtctcctcagcc

SEQ ID NO: 9 - protein, 119 aa, Homo sapiens
LESGGGVVQPGRSLRLSCAASGFTFSTYGMHWVRQAPGKGLEWVAVVSYDGTKEYYSDSVKGRFTISRDNSKKMVYLQMNSLRAEDTAVYYCAKEPPRRGFYWYFDYWGQGTLVTVSSA

SEQ ID NO: 10 - protein, 8 aa, Homo sapiens
GFTFSTYG

SEQ ID NO: 11 - protein, 8 aa, Homo sapiens
VSYDGTKE

SEQ ID NO: 12 - protein, 15 aa, Homo sapiens
AKEPPRRGFYWYFDY

SEQ ID NO: 13 - DNA, 318 bp, Homo sapiens
gagctcacgcagtctccatcctccctgtctgcatctgttggagacagagtcaccatcacttgccgggcgagtcaggacattagcaattatttagcctggtttcagcagagacccgggaaagttcctaatctcctcatctatgctgcatccactttgcaatcaggggtcccatctcgattcagtggcagtggatctggaacagaattcactctcaccatcagtagtctgcagcctgaagatggtgcaagttattactgtcaaaagtatgacggtgccccttggacgttcggccaggggaccaaggtggaaatcaaacga

SEQ ID NO: 14 - protein, 106 aa, Homo sapiens
ELTQSPSSLSASVGDRVTITCRASQDISNYLAWFQQRPGKVPNLLIYAASTLQSGVPSRFSGSGSGTEFTLTISSLQPEDGASYYCQKYDGAPWTFGQGTKVEIKR

SEQ ID NO: 15 - protein, 267 aa, Artificial
  DDF-RuV1 FAB
MKYLLPTAAAGLLLLAAQPAMAQVKLLEQSGAEVKKPGENLKISCEASGYNFVNHWIGWVRQMPGRGLEWMGRIYPGDSETRFSPSFQGQVTISVDKTLSTASLQWNSLKTSDSATYYCVTLGRWSSWQGGALSWGQGTRVIVSSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSDYKDDDDKHHHHHH

SEQ ID NO: 16 - protein, 240 aa, Artificial
  DDF-RuV1 FAB
MKYLLPTAAAGLLLLAAQPAMAELTQSPDSLAVSLGERATINCKSSQSILSTSKNKNYLAWYQQKPGQPPKLLIYWASTREDGVPDRFSGSGAGTDFSLTISSLQAEDVAVYYCQQRRSWPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 266 aa, Artificial
  DDF-RuV2 FAB
MKYLLPTAAAGLLLLAAQPAMAQVKLLESGGGVVQPGRSLRLSCAASGFTFSTYGMHWVRQAPGKGLEWVAVVSYDGTKEYYSDSVKGRFTISRDNSKKMVYLQMNSLRAEDTAVYYCAKEPPRRGFYWYFDYWGQGTLVTVSSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSDYKDDDDKHHHHHH

SEQ ID NO: 18 - protein, 234 aa, Artificial
  DDF-RuV2 FAB
MKYLLPTAAAGLLLLAAQPAMAELTQSPSSLSASVGDRVTITCRASQDISNYLAWFQQRPGKVPNLLIYAASTLQSGVPSRFSGSGSGTEFTLTISSLQPEDGASYYCQKYDGAPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 30 bp, Artificial
  PCR  PRIMER
gtggtacctttctattctctcgagcactct

SEQ ID NO: 20 - DNA, 27 bp, Artificial
  PCR PRIMER
agtttcggccgaaccacragttccacc

SEQ ID NO: 21 - DNA, 21 bp, Artificial
  PCR PRIMER
gcaattcctttagtggtacct

SEQ ID NO: 22 - DNA, 21 bp, Artificial
  PCR PRIMER
gccctcatagttagcgtaacg

SEQ ID NO: 23 - DNA, 51 bp, Artificial
  CLONING LINKER
tcgaggtaccgtatccatatgatgttccagattatgctactacggccgaaa

SEQ ID NO: 24 - DNA, 51 bp, Artificial
  CLONING LINKER
ctagtttcggccgtagtagcataatctggaacatcatatggataaggtacc

SEQ ID NO: 25 - DNA, 180 bp, Artificial
  CODING FOR N-TERMINAL pDDcXSmer-orcp3 library
  misc_feature (91)..(92): n is a, c, g, or t
  misc_feature (93)..(93): k is g or t
  misc_feature (94)..(95): n is a, c, g, or t
  misc_feature (96)..(96): k is g or t
  misc_feature (97)..(98): n is a, c, g, or t
  misc_feature (99)..(99): k is g or t
  misc_feature (100)..(101): n is a, c, g, or t
  misc_feature (102)..(102): k is g or t
  misc_feature (103)..(104): n is a, c, g, or t
  misc_feature (105)..(105): k is g or t
  misc_feature (106)..(107): n is a, c, g, or t
  misc_feature (108)..(108): k is g or t
  misc_feature (109)..(110): n is a, c, g, or t
  misc_feature (111)..(111): k is g or t
  misc_feature (112)..(113): n is a, c, g, or t
  misc_feature (114)..(114): k is g or t
  misc_feature (115)..(116): n is a, c, g, or t
  misc_feature (117)..(117): k is g or t
  misc_feature (118)..(119): n is a, c, g, or t
  misc_feature (120)..(120): k is g or t
  misc_feature (121)..(122): n is a, c, g, or t
  misc_feature (123)..(123): k is g or t
  misc_feature (124)..(125): n is a, c, g, or t
  misc_feature (126)..(126): k is g or t
atgaaatacctattgcctacggcagccgctggattgttattactcgctgcccaaccagccatggcccaggtgaaactgctcgagcactctnnknnknnknnknnknnknnknnknnknnknnknnkggtggaactagtggccaggccggccagggtggcggtggctctccattcgtttgt

SEQ ID NO: 26 - DNA, 207 bp, Artificial
  CODING N-TERMINAL pDDcKE12mer-orcp3  LIBRARY
  misc_feature (106)..(107): n is a, c, g, or t
  misc_feature (108)..(108): k is g or t
  misc_feature (109)..(110): n is a, c, g, or t
  misc_feature (111)..(111): k is g or t
  misc_feature (112)..(113): n is a, c, g, or t
  misc_feature (114)..(114): k is g or t
  misc_feature (115)..(116): n is a, c, g, or t
  misc_feature (117)..(117): k is g or t
  misc_feature (118)..(119): n is a, c, g, or t
  misc_feature (120)..(120): k is g or t
  misc_feature (121)..(122): n is a, c, g, or t
  misc_feature (123)..(123): k is g or t
  misc_feature (124)..(125): n is a, c, g, or t
  misc_feature (126)..(126): k is g or t
  misc_feature (127)..(128): n is a, c, g, or t
  misc_feature (129)..(129): k is g or t
  misc_feature (130)..(131): n is a, c, g, or t
  misc_feature (132)..(132): k is g or t
  misc_feature (133)..(134): n is a, c, g, or t
  misc_feature (135)..(135): k is g or t
  misc_feature (136)..(137): n is a, c, g, or t
  misc_feature (138)..(138): k is g or t
  misc_feature (139)..(140): n is a, c, g, or t
  misc_feature (141)..(141): k is g or t
atgaaatacctattgcctacggcagccgctggattgttattactcgctgcccaaccagccatggcccaggtgaaactgctcgaggtacctttctattctcactctnnknnknnknnknnknnknnknnknnknnknnknnkggtggaggttcggccgaaactagtggccaggccggccagggtggaggtggctctccattcgtttgt

SEQ ID NO: 27 - DNA, 36 bp, Artificial
  CODING PEPTIDE IN pDDcXSmer-orcp3/cp8 library
ctgccgcttattcagaggtcttctattcagtggacg

SEQ ID NO: 28 - protein, 12 aa, Artificial
  PEPTIDE IN pDDcXSmer-orcp3/cp8 library
LPLIQRSSIQWT

SEQ ID NO: 29 - DNA, 30 bp, Artificial
  CODING PEPTIDE IN pDDcXSmer-orcp3/cp8 library
actaatctggtgaagcagggtccgttgact

SEQ ID NO: 30 - protein, 10 aa, Artificial
  PEPTIDE IN pDDcXSmer-orcp3/cp8 library
TNLVKQGPLT

SEQ ID NO: 31 - DNA, 27 bp, Artificial
  CODING PEPTIDE IN pDDcXSmer-orcp3/cp8 library
atttatgcgatgaagaaggggccggat

SEQ ID NO: 32 - protein, 9 aa, Artificial
  PEPTIDE IN pDDcXSmer-orcp3/cp8 library
IYAMKKGPD

SEQ ID NO: 33 - DNA, 21 bp, Artificial
  CODING PEPTIDE IN pDDcXSmer-orcp3/cp8 library
tcgaatcatccgtggtagatg

SEQ ID NO: 34 - protein, 7 aa, Artificial
  PEPTIDE IN pDDcXSmer-orcp3/cp8 library
SNHPWQM

SEQ ID NO: 35 - DNA, 36 bp, Artificial
  CODING PEPTIDE IN pDDcKE12mer-orcp3/cp8
gagcatcagtggcattggtcttcgcgtgttgttcgt

SEQ ID NO: 36 - protein, 12 aa, Artificial
  PEPTIDE IN  pDDcKE12mer-orcp3/cp8
EHQWHWSSRVVR

SEQ ID NO: 37 - DNA, 36 bp, Artificial
  CODING PEPTIDE IN pDDcKE12mer-orcp3/cp8
cagactccgctgtagactattcgggtgattcctggg

SEQ ID NO: 38 - protein, 12 aa, Artificial
  PEPTIDE IN pDDcKE12mer-orcp3/cp8
QTPLQTIRVIPG

SEQ ID NO: 39 - DNA, 36 bp, Artificial
  CODING PEPTIDE IN pDDcKE12mer-orcp3/cp8
ttgaatactttgacgtctcttagtctgccgtaggcg

SEQ ID NO: 40 - protein, 12 aa, Artificial
  CODING PEPTIDE IN pDDcKE12mer-orcp3/cp8 library
LNTLTSLSLPQA

SEQ ID NO: 41 - DNA, 36 bp, Artificial
  CODING PEPTIDE IN pDDcKE12mer-orcp3/cp8 library
aagcctaatatgtagaattctcatattggttcggcg

SEQ ID NO: 42 - protein, 12 aa, Artificial
  CODING SEQUENCE IN pDDcKE12mer-orcp3/cp8 library
KPNMQNSHIGSA